(19) United States
(12) Patent Application Publication
(10) Pub. No.: US 2021/0292434 A1
(43) Pub. Date: Sep. 23, 2021

(54) NOVEL STABLE HIGH-CONCENTRATION FORMULATION FOR ANTI-FXIa ANTIBODIES

(71) Applicant: Bayer Aktiengesellschaft, Leverkusen (DE)

(72) Inventors: Niklas GOMBERT, Wuppertal (DE); Marieke VEURINK, Hamburg (DE); Alexander KLAK, Köln (DE); Stefan Christian SCHNEID, Düsseldorf (DE); Stefan HEKE, Wuppertal (DE); Matthias PLITZKO, Neuenburg (DE)

(73) Assignee: Bayer Aktiengesellschaft, Leverkusen (DE)

(21) Appl. No.: 17/257,828

(22) PCT Filed: Jul. 5, 2019
(86) PCT No.: PCT/EP2019/068106

§ 371 Date: Jan. 4, 2021

(30) Foreign Application Priority Data
Jul. 5, 2018 (EP) PCT/EP2018/068250

(51) Int. Cl. C07K 16/36; A61K 47/10; A61K 47/18; A61K 47/26; A61K 9/19; A61K 9/08

(52) U.S. Cl. CPC C07K 16/36; A61K 47/10; A61K 47/183; C07K 2317/94; A61K 9/19; A61K 9/08; A61K 47/26

(57) ABSTRACT

The present invention refers to novel liquid pharmaceutical high-concentration formulations particularly suitable for subcutaneous administration comprising human antibodies against coagulation factor FXIa as active ingredient, especially those described in WO2013167669, which are stable as liquid formulations over a long period. The invention also refers to lyophilizates of the specified liquid formulation with reduced reconstitution time and also to the use of these formulations in the therapy and prophylaxis of thrombotic or thromboembolic disorder.

### INTRODUCTION

The present invention refers to novel liquid pharmaceutical high-concentration formulations particularly suitable for subcutaneous administration comprising human antibodies against coagulation factor FXIa as active ingredient, especially those described in WO2013167669, which are stable as liquid formulations over a long period. The invention also refers to lyophilizates of the specified liquid formulation with reduced reconstitution time and also to the use of these formulations in the therapy and prophylaxis of thrombotic or thromboembolic disorders.

Blood coagulation is a protective mechanism of the organism which helps to be able to “seal” defects in the wall of the blood vessels quickly and reliably. Thus, loss of blood can be avoided or kept to a minimum. Haemostasis after injury of the blood vessels is affected mainly by the coagulation system in which an enzymatic cascade of complex reactions of plasma proteins is triggered. Numerous blood coagulation factors are involved in this process, each of which factors converts, on activation, the respectively next inactive precursor into its active form. At the end of the cascade comes the conversion of soluble fibrinogen into insoluble fibrin, resulting in the formation of a blood clot. In blood coagulation, traditionally the intrinsic and the extrinsic system, which end in a final joint reaction path, are distinguished.

Coagulation factor XIa is a central component of the transition from initiation to amplification and propagation of coagulation: in positive feedback loops, thrombin activates, in addition to factor V and factor VIII, also factor XI to factor XIa, whereby factor IX is converted into factor IXa, and, via the factor IXa/factor VIIIa complex generated in this manner, the factor X is activated and thrombin formation is in turn therefore highly stimulated leading to strong thrombus growth and stabilizing the thrombus. Anti-FXIa antibodies are known in the prior art as anticoagulants, i.e. substances for inhibiting or preventing blood coagulation (see WO2013167669).

Therapeutic proteins such as, for example, human monoclonal antibodies are generally administered by injection as liquid pharmaceutical formulations. Since many therapeutically effective human monoclonal antibodies have unfavourable properties such as low stability or a tendency to aggregation, it is necessary to modulate these unfavourable properties by suitable pharmaceutical formulation. An aggregate or denatured antibody may have, for example, a low therapeutic efficacy. An aggregate or denatured antibody may also provoke undesired immunological reactions. Stable pharmaceutical formulations of proteins should also be suitable to prevent chemical instabilities. Chemical instability of proteins may lead to degradation or fragmentation and thus reduced efficacy or even to toxic side effects. The formation or generation of all types of low-molecular-weight fragments should therefore be avoided or at least minimized. These are all factors which may affect the safety of a preparation and therefore must be taken into account. Furthermore, a low viscosity is of fundamental when using syringes or pumps since this keeps the force required low and therefore increases the injectability. A low viscosity is also fundamental during production, for example, enabling the precise filling of a preparation. The therapeutic use of a human monoclonal antibody, however, often requires the use of high antibody concentration, which often leads to problems with high viscosity. In their overview article, Daugherty and Mrsny (Adv Drug Deliv Rev. 2006; 58(5-6):686-706) discuss this and other problems which can occur in the liquid pharmaceutical formulation of monoclonal antibodies.

For subcutaneous (s.c.) injection, special requirements on a formulation have to be considered additionally. Compared to intravenous application the injection volume is limited for single injection by syringes as formulations in delivery volumes greater than 1-2 milliliters are not well tolerated. This leads to a necessity of higher antibody concentration to deliver the same dose. This means that the antibody has to reach concentrations of about 100 mg/ml or more. Highly concentrated protein formulations can pose many challenges to the manufacturability and administration of protein therapeutics. Furthermore, convenient application through a small needle is demanded to assure patient acceptance and compliance. For highly concentrated antibody formulations both attributes rival as with increasing antibody concentration viscosity rises and impedes administration.

A liquid low concentration formulation for anti-FXIa antibodies suitable for intravenous (i.v.) application which allows a higher injection volume compared to subcutaneous application is described in patent application PCT/EP2018/050951. This low concentration formulation comprises 10-40 mg/ml anti-FXIa antibody and a histidine/glycine buffer system comprising 5-10 mM histidine and 130-200 mM glycine, wherein the formulation has a pH of 5.7-6.3. Considering a limited application volume of ≤2 ml for subcutaneous application, the low concentration formulation as described in PCT/EP2018/050951 is not suitable for administration of the intended therapeutically relevant dose. An increase of anti-FXIa antibody concentration to about 100 mg/ml or more is inevitable and obvious. However, increasing the concentration of anti-FXIa antibody in the histidine/glycine buffer system described in PCT/EP2018/050951 resulted in an exponential increase in viscosity of the solution to unacceptable values.

Various methods have been proposed to overcome the challenges associated with high-concentration dosage forms. For example, to address the stability problem associated with high-concentration antibody formulations, the antibody is often lyophilized, and then reconstituted shortly before administration. Reconstitution is generally not optimal, since it adds an additional, sometimes time-consuming, step to the administration process, and could introduce contaminants to the formulation. Additionally, even reconstituted antibodies can suffer from aggregation and high viscosity. Therefore, liquid formulations which are stable over a long period would be advantageous.

Several liquid high-concentration formulations for proteins and antibodies using different excipients to lower the viscosity are known in the art. WO2009043049, for example, describes the use of an excipient selected from the group consisting of creatine, creatinine, carnitine and mixtures thereof for reducing the viscosity of liquid pharmaceutical protein formulation with a concentration of at least 70 mg/ml protein. Whereas, in WO2016065181 the use of various n-acetyl amino acids for reducing the viscosity of high-concentration formulations is described.

Arginine is known as viscosity reducing excipient. But until now only high-concentration protein formulations are known wherein high amounts of arginine, or high amounts of arginine and histidine were necessary to provide sufficient viscosity reduction. US20150239970 describes the use of high amounts of arginine (more than 150 mM) for a liquid high-concentration formulation of an anti-IL-6 antibody. Whereas, in U.S. Pat. No. 8,703,126 the use of about 150-200 mM salt or buffer derived from arginine or histidine is described.

There exists a need for highly concentrated liquid formulations of anti-FXIa antibodies which comprise a low fraction of aggregates and degradation products, are stable as a liquid over a long period, without the need to be lyophilized, and have minimal viscosity. Furthermore, pH of formulations for s.c. application should be in the range from 4.7 to 7.4 and osmolarity should not exceed a range of 240 to 400 mOsm/kg.

The present invention addresses the need mentioned above and provides liquid high concentration pharmaceutical formulations comprising about 100 mg/ml or more anti-FXIa antibodies and low amounts of aggregates and degradation products, which are stable as liquids over a long period. These formulations also have a low viscosity and may therefore be simply administered to patients, even by subcutaneous injection, for example by means of syringes, pen devices, autoinjectors or any other devices known in the art. The liquid high-concentration anti-FXIa formulation may also be lyophilized, preferably by a spray-freezing-based method as described in patent application EP17170483.6 which provides for significant shorter reconstitution times than conventional freeze-drying methods.

The invention provides high-concentration pharmaceutical formulations with low viscosity, especially suitable for subcutaneous application, comprising about 100 mg/ml or more anti-FXIa antibodies and a triple buffer system at a low pH of 4.7-5.3, comprising histidine, glycine, and arginine, wherein low amounts of arginine are sufficient to reduce viscosity and which are stable as liquids over a long period.

### BRIEF DESCRIPTION OF THE FIGURES

FIG. 1 schematically shows an apparatus for the freeze-drying method leading to freeze-dried pellets with reduced reconstitution time (Method 3).

FIG. 2 graphically depicts the temperature and pressure profile measured over time during conventional freeze-drying (Method 1) of the antibody solution.

FIG. 3 graphically depicts the temperature and pressure profile measured over time during freezing and drying of the antibody solution according to Method 2 (as described in WO2006/008006).

FIG. 4 graphically depicts the temperature profile in the cooling tower measured over time during processing of the antibody solution according to the freeze-drying method as described herein (Method 3).

FIG. 5 graphically depicts the temperature and pressure profile measured over time during freezing and drying of the antibody solution according to the freeze-drying method as described herein (Method 3)

FIG. 6 shows Scanning Electron Microscopy (SEM) pictures of a pellet produced according to the freeze-drying method as described herein (Method 3)

FIG. 7 shows Scanning Electron Microscopy (SEM) pictures of a lyophilizate produced according to conventional freeze-drying (Method 1)

FIG. 8 shows Scanning Electron Microscopy (SEM) pictures of a lyophilizate produced according to the freeze-drying process disclosed in WO2006/008006 (Method 2).

### DESCRIPTION OF THE INVENTION

In one embodiment, the liquid pharmaceutical formulation comprises 5-30 mM histidine, 20-100 mM glycine and less than 150 mM arginine. In a preferred embodiment, the liquid pharmaceutical formulation comprises 10-20 mM histidine, 25-75 mM glycine and 50-75 mM arginine. In a particularly preferred embodiment, the liquid pharmaceutical formulation comprises 20 mM histidine, 50 mM glycine and 50 mM arginine. Furthermore, the liquid pharmaceutical formulation has a pH of 4.7-6.0. In a preferred embodiment, the liquid pharmaceutical formulation has a pH of 4.7-5.3. In a particularly preferred embodiment, the liquid pharmaceutical formulation has a pH of 5. The liquid pharmaceutical formulation according to the invention comprises anti-FXIa antibodies at concentrations of about 100 mg/ml or more. In a preferred embodiment, the anti-FXIa antibody is present at concentrations of about 100-300 mg/ml. In a particularly preferred embodiment, the anti-FXIa antibody has a concentration of 135-165 mg/ml, most preferred of about 150 mg/ml. In a further particularly preferred embodiment, the anti-FXIa antibody has a concentration of about 100 mg/ml. In all embodiments, the anti-FXIa antibody is particularly preferably 076D-M007-H04-CDRL3-N110D.

The liquid pharmaceutical formulation may also comprise a stabilizer.

Stabilizers are sugars for example. “Sugars” refers to a group of organic compounds which are water-soluble and are divided among monosaccharides, disaccharides and polyols. A preferred sugar is a non-reducing disaccharide, particular preference being given to trehalose. In one embodiment, the stabilizer is present to an extent of 1-10% weight to volume (w/v), preferably to an extent of 3-7% (w/v) and particularly preferably to an extent of 5% (w/v). In a preferred embodiment, trehalose dihydrate is present to an extent of 1-10% weight to volume (w/v), preferably to an extent of 3-7% (w/v) and particularly preferably to an extent of 5% (w/v).

The liquid pharmaceutical formulation may also comprise a surfactant. The term “surfactant” refers to any detergent having a hydrophilic and a hydrophobic region and includes non-ionic, cationic, anionic and zwitterionic detergents. Preferred detergents may be selected from the group consisting of polyoxyethylene sorbitan monooleate (also known as polysorbate 80 or TWEEN 80), polyoxyethylene sorbitan monolaurate (also known as polysorbate 20 or TWEEN 20), poloxamer 188 (a copolymer of polyoxyethylene and polyoxypropylene) and N-laurylsarcosine. For the compositions disclosed, preference is given to a non-ionic surfactant. Particular preference is given to the use of polysorbate 80, polysorbate 20 or poloxamer 188 for the compositions of the present invention. The surfactant may be used at a concentration of 0.005% to 0.5% (w/v), preference being given to a concentration range of 0.01% to 0.2% (w/v). Particular preference is given to using a surfactant agent concentration of 0.05%-0.1% (w/v). Especially preferred is the use of polysorbate 80 at a concentration of 0.1% (w/v). Further especially preferred is the use of polysorbate 20 or poloxamer 188 at a concentration of 0.05% (w/v).

Preservatives or other additives, fillers, stabilizers or carriers may optionally be added to the liquid pharmaceutical formulations according to the invention. Suitable preservatives are, for example, octadecyldimethylbenzylammonium chloride, hexamethonium chloride, and aromatic alcohols such as phenol, parabens or m-cresol.

Further pharmaceutically acceptable additives, stabilizers or carriers are described, for example, in Remington's Science And Practice of Pharmacy (22nd edition, Loyd V. Allen, Jr, editor. Philadelphia, Pa.: Pharmaceutical Press. 2012).

The invention therefore provides a liquid pharmaceutical high-concentration formulation comprising about 100 mg/ml or more of the anti-FXIa antibody 076D-M007-H04-CDRL3-N110D and a histidine/glycine/arginine buffer system, wherein the formulation comprises 5-30 mM histidine, 20-100 mM glycine and less than 150 mM arginine, preferably 10-20 mM histidine, 25-75 mM glycine and 50-75 mM arginine and has a pH of 4.7-5.3, preferably pH 5. This results in a high-concentration formulation of the antibody 076D-M007-H04-CDRL3-N110D at low viscosity, sufficient stabilization and low aggregation which is stable as liquid formulation, but also enables optional lyophilization of the formulation.

One embodiment according to the invention is a liquid pharmaceutical formulation comprising about 100 mg/ml of the anti-FXIa antibody 076D-M007-H04-CDRL3-N110D and a histidine/glycine/arginine buffer system, wherein the formulation comprises 5-30 mM histidine, 20-100 mM glycine and less than 150 mM arginine, preferably 10-20 mM histidine, 25-75 mM glycine and 50-75 mM arginine and has a pH of 4.7-5.3, preferably pH 5.

One embodiment according to the invention is a liquid pharmaceutical formulation comprising

anti-FXIa antibody M007-H04-CDRL3-N110D at a concentration of about 100 mg/ml or more,

5-30 mM histidine, preferably 10-20 mM histidine,

20-100 mM glycine, preferably 25-75 mM glycine and

less than 150 mM arginine, preferably 50-75 mM arginine,

wherein the formulation has a pH of 4.7-5.3, preferably a pH of 5. The formulation optionally comprises further ingredients selected from the group consisting of surfactant, preservatives, carriers and stabilizers.

One embodiment according to the invention is a liquid pharmaceutical formulation comprising

anti-FXIa antibody 076D-M007-H04-CDRL3-N110D at a concentration of about 100 mg/ml or more,

5-30 mM histidine, preferably 10-20 mM histidine,

20-100 mM glycine, preferably 25-75 mM glycine and

less than 150 mM arginine, preferably 50-75 mM arginine, 1-10% (w/v) stabilizer, preferably 3-7% (w/v) trehalose dihydrate,

wherein the formulation has a pH of 4.7-5.3, preferably a pH of 5. The formulation optionally comprises further ingredients selected from the group consisting of surfactant, preservatives, carriers and stabilizers.

In one embodiment, the liquid pharmaceutical formulation comprises polysorbate 80, polysorbate 20 or poloxamer 188 as surfactant at a concentration of 0.005% to 0.5% (w/v), preferably 0.01% to 0.2% (w/v).

A preferred embodiment is a liquid pharmaceutical formulation comprising

anti-FXIa antibody 076D-M007-H04-CDRL3-N110D at a concentration of about 100 mg/ml or more,

10-20 mM histidine, 25-100 mM glycine and 50-75 mM arginine,

3-7% (w/v) trehalose dihydrate, and

polysorbate 80, polysorbate 20 or poloxamer 188 at a concentration of 0.01% to 0.2% (w/v),

wherein the formulation has a pH of 4.7-5.3, preferably a pH of 5. The formulation optionally comprises further ingredients selected from the group consisting of preservatives, carriers and stabilizers.

A preferred embodiment is a liquid pharmaceutical formulation comprising

anti-FXIa antibody 076D-M007-H04-CDRL3-N110D at a concentration of about 100 mg/ml or more,

10-20 mM histidine, 25-100 mM glycine and 50-75 mM arginine,

3-7% (w/v) trehalose dihydrate, and

polysorbate 80 at a concentration of 0.01% to 0.2% (w/v),

wherein the formulation has a pH of 4.7-5.3, preferably a pH of 5. The formulation optionally comprises further ingredients selected from the group consisting of preservatives, carriers and stabilizers.

A further preferred embodiment is a liquid pharmaceutical formulation comprising

anti-FXIa antibody 076D-M007-H04-CDRL3-N110D at a concentration of about 100 mg/ml or more,

10-20 mM histidine, 25-100 mM glycine and 50-75 mM arginine,

3-7% (w/v) trehalose dihydrate, and

polysorbate 20 or poloxamer 188 at a concentration of 0.01% to 0.2% (w/v),

wherein the formulation has a pH of 4.7-5.3, preferably a pH of 5. The formulation optionally comprises further ingredients selected from the group consisting of preservatives, carriers and stabilizers.

A particularly preferred embodiment is a liquid pharmaceutical formulation comprising

anti-FXIa antibody 076D-M007-H04-CDRL3-N110D at a concentration of about 100 mg/ml or more,

20 mM histidine, 50 mM glycine, and 50 mM arginine,

5% (w/v) trehalose dihydrate, and

polysorbate 80 at a concentration of 0.1% (w/v),

wherein the formulation has a pH of 5. The formulation optionally comprises further ingredients selected from the group consisting of preservatives, carriers and stabilizers.

A further particularly preferred embodiment is a liquid pharmaceutical formulation comprising

anti-FXIa antibody 076D-M007-H04-CDRL3-N110D at a concentration of about 100 mg/ml or more,

20 mM histidine, 50 mM glycine, and 50 mM arginine,

5% (w/v) trehalose dihydrate, and

polysorbate 20 at a concentration of 0.05% (w/v),

wherein the formulation has a pH of 5. The formulation optionally comprises further ingredients selected from the group consisting of preservatives, carriers and stabilizers.

A further particularly preferred embodiment is a liquid pharmaceutical formulation comprising

anti-FXIa antibody 076D-M007-H04-CDRL3-N110D at a concentration of about 100 mg/ml or more,

20 mM histidine, 50 mM glycine, and 50 mM arginine,

5% (w/v) trehalose dihydrate, and

poloxamer 188 at a concentration of 0.05% (w/v),

wherein the formulation has a pH of 5. The formulation optionally comprises further ingredients selected from the group consisting of preservatives, carriers and stabilizers.

A further particularly preferred embodiment is a liquid pharmaceutical formulation comprising

anti-FXIa antibody 076D-M007-H04-CDRL3-N110D at a concentration of about 150 mg/ml or more,

20 mM histidine, 50 mM glycine, and 50 mM arginine,

5% (w/v) trehalose dihydrate, and

polysorbate 80 at a concentration of 0.1% (w/v),

wherein the formulation has a pH of 5. The formulation optionally comprises further ingredients selected from the group consisting of preservatives, carriers and stabilizers.

A further particularly preferred embodiment is a liquid pharmaceutical formulation comprising

anti-FXIa antibody 076D-M007-H04-CDRL3-N110D at a concentration of about 150 mg/ml or more,

20 mM histidine, 50 mM glycine, and 50 mM arginine,

5% (w/v) trehalose dihydrate, and

polysorbate 20 at a concentration of 0.05% (w/v),

wherein the formulation has a pH of 5. The formulation optionally comprises further ingredients selected from the group consisting of preservatives, carriers and stabilizers.

A further particularly preferred embodiment is a liquid pharmaceutical formulation comprising

anti-FXIa antibody 076D-M007-H04-CDRL3-N110D at a concentration of about 150 mg/ml or more,

20 mM histidine, 50 mM glycine, and 50 mM arginine,

5% (w/v) trehalose dihydrate, and

poloxamer 188 at a concentration of 0.05% (w/v),

wherein the formulation has a pH of 5. The formulation optionally comprises further ingredients selected from the group consisting of preservatives, carriers and stabilizers.

The anti-FXIa antibody to be used in accordance with the present invention is capable of binding to the activated form of plasma factor XI, FXIa. Preferably, the anti-FXIa antibody specifically binds to FXIa. Preferably, the anti-FXIa antibody is capable of inhibiting platelet aggregation and associated thrombosis. Preferably, antibody mediated inhibition of platelet aggregation does not compromise platelet-dependent primary hemostasis. In the context of the present invention the term “without compromising hemostasis” means that the inhibition of coagulation factor XIa does not lead to unwanted and measurable bleeding events.

As used herein, “coagulation factor XIa,” “factor XIa”, or “FXIa” refers to any FXIa from any mammalian species that expresses the zymogen factor XI. For example, FXIa can be human, non-human primate (such as baboon), mouse, dog, cat, cow, horse, pig, rabbit, and any other species expressing the coagulation factor XI involved in the regulation of blood flow, coagulation, and/or thrombosis.

As used herein, an antibody “binds specifically to,” is “specific to/for” or “specifically recognizes” an antigen (here, FXIa) if such antibody is able to discriminate between such antigen and one or more reference antigen(s), since binding specificity is not an absolute, but a relative property. In its most general form (and when no defined reference is mentioned), “specific binding” is referring to the ability of the antibody to discriminate between the antigen of interest and an unrelated antigen, as determined, for example, in accordance with one of the following methods. Such methods comprise, but are not limited to Western blots, ELISA-, RIA-, ECL-, IRMA-tests and peptide scans. For example, a standard ELISA assay can be carried out. The scoring may be carried out by standard colour development (e.g. secondary antibody with horseradish peroxide and tetramethyl benzidine with hydrogenperoxide). The reaction in certain wells is scored by the optical density, for example, at 450 ran. Typical background (=negative reaction) may be 0.1 OD; typical positive reaction may be 1 OD. This means the difference positive/negative can be more than 10-fold. Typically, determination of binding specificity is performed by using not a single reference antigen, but a set of about three to five unrelated antigens, such as milk powder, BSA, transferrin or the like. However, “specific binding” also may refer to the ability of an antibody to discriminate between the target antigen and one or more closely related antigen(s), e.g., homologs, which are used as reference points. For instance, the antibody may have at least at least 1.5-fold, 5 2-fold, 5-fold 10-fold, 100-fold, 103-fold, 104-fold, 105-fold, 106-fold or greater relative affinity for the target antigen as compared to the reference antigen. Additionally, “specific binding” may relate to the ability of an antibody to discriminate between different parts of its target antigen, e.g. different domains or regions of FXIa.

“Affinity” or “binding affinity” KD are often determined by measurement of the equilibrium association constant (ka) and equilibrium dissociation constant (kd) and calculating the quotient of kd to ka (KD=kd/ka). The term “immunospecific” or “specifically binding” preferably means that the antibody binds to the coagulation factor XIa with an affinity KD of lower than or equal to 106M (monovalent affinity). The term “high affinity” means that the KD that the antibody binds to the coagulation factor XIa with an affinity KD of lower than or equal to 107M (monovalent affinity). Such affinities may be readily determined using conventional techniques, such as by equilibrium dialysis; by using the BIAcore 2000 instrument, using general procedures outlined by the manufacturer; by radioimmunoassay using radio labeled target antigen; or by another method known to the skilled artisan. The affinity data may be analyzed, for example, by the method described in [Kaufman R J, Sharp P A. (1982) Amplification and expression of sequences cotransfected with a modular dihydrofolate reductase complementary dna gene. [J Mol Biol. 159:601-621].

As used herein, the term “antibody” includes immunoglobulin molecules (e.g., any type, including IgG, IgEl IgM, IgD, IgA and IgY, and/or any class, including, IgGI, IgG2, IgG3, IgG4, IgAI and IgA2) isolated from nature or prepared by recombinant means and includes all conventionally known antibodies and functional fragments thereof. The term “antibody” also extends to other protein scaffolds that are able to orient antibody CDR inserts into the same active binding conformation as that found in natural antibodies such that binding of the target antigen observed with these chimeric proteins is maintained relative to the binding activity of the natural antibody from which the CDRs were derived.

A “functional fragment” or “antigen-binding antibody fragment” of an antibody/immunoglobulin hereby is defined as a fragment of an antibody/immunoglobulin (e.g., a variable region of an IgG) that retains the antigen-binding region. An “antigen-binding region” of an antibody typically is found in one or more hypervariable region(s) of an antibody, i.e., the CDR-I, -2, and/or -3 regions; however, the variable “framework” regions can also play an important role in antigen binding, such as by providing a scaffold for the CDRs. Preferably, the “antigen-binding region” comprises at least amino acid residues 4 to 103 of the variable light (VL) chain and 5 to 109 of the variable heavy (VH) chain, more preferably amino acid residues 3 to 107 of VL and 4 to 111 of VH, and particularly preferred are the complete VL and VH chains (amino acid positions 1 to 109 of VL and 1 to 113 of VH; numbering according to WO 97/08320). A preferred class of immunoglobulins for use in the present invention is IgG.

“Functional fragments” of the invention include Fab, Fab1, F(ab′)2, and Fv fragments; diabodies; linear antibodies; single-chain antibody molecules (scFv); and multispecific antibodies formed from antibody fragments, disulfide-linked Fvs (sdFv), and fragments comprising a VL or VH domain, which are prepared from intact immunoglobulins or prepared by recombinant means.

Antigen-binding antibody fragments may comprise the variable region(s) alone or in combination with the entirety or a portion of the following: hinge region, CHI, CH2, CH3 and CL domains. Also included in the invention are antigen-binding antibody fragments comprising any combination of variable region(s) with a hinge region, CHI, CH2, CH3 and CL domain.

The antibody and/or antigen-binding antibody fragment may be monospecific (e.g. monoclonal), bispecific, trispecific or of greater multi specificity. Preferably, a monoclonal antibody is used.

The term “monoclonal antibody” as used herein refers to an antibody obtained from a population of substantially homogeneous antibodies, i.e., the individual antibodies comprising the population are identical except for possible naturally occurring mutations that may be present in minor amounts. Monoclonal antibodies are highly specific, being directed against a single antigenic site. Furthermore, in contrast to conventional (polyclonal) antibody preparations that typically include different antibodies directed against different determinants (epitopes), each monoclonal antibody is directed against a single determinant on the antigen. In addition to their specificity, the monoclonal antibodies are advantageous in that they are synthesized by the homogeneous culture, uncontaminated by other immunoglobulins with different specificities and characteristics. The modifier “monoclonal” indicates the character of the antibody as being obtained from a substantially homogeneous population of antibodies, and is not to be construed as requiring production of the antibody by any particular method.

The antibody or antigen-binding antibody fragment may for instance be human, humanized, murine (e.g., mouse and rat), donkey, sheep, rabbit, goat, guinea pig, camelid, horse, or chicken. Preferably, a human or humanized anti-FXIa antibody is used.

As used herein, “human” antibodies include antibodies having the amino acid sequence of a human immunoglobulin and include antibodies isolated from human immunoglobulin libraries, from human B cells, or from animals transgenic for one or more human immunoglobulin as well as synthetic human antibodies.

A “humanized antibody” or functional humanized antibody fragment is defined herein as one that is (i) derived from a non-human source (e.g., a transgenic mouse which bears a heterologous immune system), which antibody is based on a human germline sequence; or (ii) chimeric, wherein the variable domain is derived from a non-human origin and the constant domain is derived from a human origin or (iii) CDR-grafted, wherein the CDRs of the variable domain are from a nonhuman origin, while one or more frameworks of the variable domain are of human 5 origin and the constant domain (if any) is of human origin.

Suitable antibodies to be used in accordance with the present invention are for instance disclosed in WO 2013/167669. In one embodiment, the anti-FXIa antibody comprises i) SEQ ID NO: 19 for the amino acid sequence for the variable light chain domain and SEQ ID NO: 20 for the amino acid sequence for the variable heavy chain domain; or ii) SEQ ID NO SEQ ID NO: 29 for the amino acid sequence for the variable light chain domain and SEQ ID NO: 30 for the amino acid sequence for the variable heavy chain domain; or iii) SEQ ID NO: 27 for the amino acid sequence for the variable light chain domain and SEQ ID NO: 20 for the amino acid sequence for the variable heavy chain domain as disclosed in WO 2013/167669. In preferred embodiments, the anti-FXIa antibody is selected from antibodies 076D-M007-H04, 076D-M007-H04-CDRL3-N110D, and 076D-M028-H17 disclosed in WO 2013/167669. In particular preferred embodiments the anti-FXIa antibody is 076D-M007-H04-CDRL3-N110D, herein represented by SEQ ID NO: 1 for the amino acid sequence for the variable heavy chain domain and SEQ ID NO: 2 for the amino acid sequence for the variable light chain domain.

The term “pharmaceutical formulation” or “formulation” as used herein refers to a preparation which is in such form as to permit the biological activity of an active ingredient contained therein to be effective, and which contains no additional components which are unacceptably toxic to a subject to which the formulation would be administered.

As used herein, “viscosity” is a fluid's resistance to flow, and may be measured in units of centipoise (cP) or milliPascal-second (mPa*s), where 1 cP=1 mPa*s, at a given shear rate. Viscosity may be measured by using a viscometer, e.g., a small sample viscometer as mVroc, RheoSense. Viscosity may be measured using any other methods and in any other units known in the art (e.g. absolute, kinematic or dynamic viscosity), understanding that it is the percent reduction in viscosity afforded by use of the excipients described by the invention that is important. Regardless of the method used to determine viscosity, the percent reduction in viscosity in excipient formulations versus control formulations will remain approximately the same at a given shear rate.

As used herein, a formulation containing an amount of an excipient effective to “reduce viscosity” (or a “viscosity-reducing” amount or concentration of such excipient) means that the viscosity of the formulation in its final form for administration (if a solution, or if a powder, upon reconstitution with the intended amount of diluent) is at least 5% less than the viscosity of an appropriate control formulation, such as water, buffer, other known viscosity-reducing agents such as salt, etc. and those control formulations, for example, exemplified herein.

Similarly, a “reduced viscosity” formulation is a formulation that exhibits reduced viscosity compared to a control formulation.

The term “buffer”, as used herein, refers to a buffered solution, which pH changes only marginally after addition of acidic or basic substances. Buffered solutions contain a mixture of a weak acid and its corresponding base, or a weak base and its corresponding acid, respectively. Exemplary pharmaceutically acceptable buffers include acetate (e.g. sodium acetate), succinate (such as sodium succinate), phosphate, glutamic acid, glutamate, gluconate, histidine, glycine, citrate or other organic acid buffers. Optionally, mixtures of one or more of the aforementioned acids and bases can be used in a buffered solution. Exemplary buffer concentration of each of the aforementioned acids and bases can be from about 1 mM to about 200 mM, from about 10 mM to about 100 mM, or from about 20 mM to 50 mM, depending, for example, on the buffer and the desired tonicity (e.g. isotonic, hypertonic or hypotonic) of the formulation.

The term “buffering system”, as used herein, refers to a mixture of one or more of the aforementioned acids and bases. A preferred buffering system of this invention contains one or more amino acids. Most preferably the buffering system comprises a mixture of histidine, glycine and arginine wherein low amounts of arginine, below 150 mM, are sufficient to reduce viscosity. Preferably arginine is contained in a concentration of 50-75 mM, most preferably in a concentration of 50 mM.

In the context of this invention “% (w/v)” defines the mass concentration of a component in percent within a composition, wherein w means the mass (measured in g, mg etc.) of the component employed, and v means the final volume (measured in L, ml etc.) of the composition.

The term “patient” refers to human or animal individuals receiving a preventive or therapeutic treatment.

The term “treatment” herein refers to the use or administration of a therapeutic substance on/to a patient, or to the use or administration of a therapeutic substance on/to an isolated tissue or on/to a cell line of a patient, who is suffering from a disease, is showing a symptom of a disease, or has a predisposition to a disease, with the goal of curing, improving, influencing, stopping or alleviating the disease, its symptoms or the predisposition to the disease.

“Effective dose” describes herein the active-ingredient amount with which the desired effect can be at least partially achieved. A “therapeutically effective dose” is therefore defined as the active-ingredient amount which is sufficient to at least partially cure a disease, or to at least partially eliminate adverse effects in the patient that are caused by the disease. The amounts actually required for this purpose are dependent on the severity of the disease and on the general immune status of the patient.

Pharmaceutical compositions suitable for use in the present invention include compositions wherein the active ingredients are contained in an effective amount to achieve the intended purpose, i.e. treatment of a particular disease. The determination of an effective dose is well within the capability of those skilled in the art.

The concentration of the therapeutic protein, such as an antibody, in the formulation will depend upon the end use of the pharmaceutical formulation and can be easily determined by a person of skill in the art.

Therapeutic proteins for subcutaneous administration are frequently administered at high-concentrations. Particularly contemplated high-concentrations of therapeutic proteins (without taking into account the weight of chemical modifications such as pegylation), including antibodies, are at least about 70, 80, 90, 95, 100, 105, 110, 115, 120, 125, 130, 135, 140, 145, 150, 155, 160, 165, 175, 180, 185, 190, 195, 200, 250, 300, 350, 400, 450, or 500 mg/ml, and/or less than about 250, 300, 350, 400, 450 or 500 mg/ml. Exemplary high-concentrations of therapeutic proteins, such as antibodies, in the formulation may range from about 100 mg/ml to about 500 mg/ml. Preferably, the concentrations of the therapeutic protein according to the invention are in the range of about 100-300 mg/ml, more preferred in the range of 135-165 mg/ml, most preferred of about 150 mg/ml. A further most preferred concentration is about 100 mg/ml. In this context a concentration of “about” a given value, e.g. the upper or lower limit of a given concentration range, is to be understood as encompassing all concentration deviating up to ±10% from this given value.

The term “high-molecular-weight aggregates” (synonym: “HMW”) describes aggregates which are composed of at least two protein monomers.

The invention further provides a product which comprises one of the pharmaceutical formulations according to the invention and preferably also instructions for use. In one embodiment, the product comprises a container which comprises liquid formulations according to the invention. Useable containers are, for example, bottles, vials, tubes, cartridges, single or multi-chambered syringes or any other containers known in the art. The containers can, for example, be composed of glass or plastic. Exemplary administration devices include syringes, with or without needles, infusion pumps, jet injectors, pen devices, transdermal injectors, or other needle-free injectors. Syringes, pen devices, autoinjectors or any other devices known in the art can comprise an injection needle composed, for example, of metal. The invention further provides a kit which comprises the aforementioned pharmaceutical formulations.

In one embodiment, the container is a syringe. In a further embodiment, the syringe is pre-filled. In a further embodiment, the syringe is contained in an injection device. In a further embodiment the injection device is an autoinjector. In another embodiment, the container is a cartridge. In a further embodiment the cartridge is contained in a pen device or any other device known in the art. In another embodiment the container is a vial.

The compositions according to the invention exhibit increased stability at high antibody concentrations compared to the formulations for anti-FXIa antibodies available in the prior art. The preferred formulations are stable as liquid formulations but can also be lyophilized. The liquid pharmaceutical formulation according to the invention accordingly may also be a reconstituted lyophilizate obtained by conventional freeze-drying methods (Method 1) or by a spray-freezing-based method as for example described in WO2006/008006 (Method 2) or Method 3 as described herein. Preferably, the lyophilizate is obtained by the spray-freezing-based Method 3 as described herein which provides for freeze-dried pellets with reduced reconstitution time.

In conventional processes, freeze-drying is usually performed in standard freeze-drying chambers comprising one or more trays or shelves within a (vacuum) drying chamber. Vials can be filled with the product to be freeze-dried and arranged on these trays. These dryers typically do not have temperature controlled walls and provide non-homogeneous heat transfer to the vials placed in the dryer chamber. Especially those vials which are positioned at the edges exchange energy more intensively than those positioned in the center of the plates, due to radiant heat transfer and gas conduction in the gap between the wall of the chamber and the stack of plates/shelves. This non-uniformity of energy distribution leads to a variation of freezing and drying kinetics between the vials at the edges and those in the center, and could result in variation in the activities of the active contents of the respective vials and product yield losses. To ensure the uniformity of the final product, it is necessary to conduct extensive development and validation work both at laboratory and production scales.

WO2006/008006 A1 is concerned with a process for sterile manufacturing, including freeze-drying, storing, assaying and filling of pelletized biopharmaceutical products in final containers such as vials. The described process combines spray-freezing and freeze-drying and comprises the steps of: a) freezing droplets of the product to form pellets, whereby the droplets are formed by passing a solution of the product through frequency assisted nozzles and pellets are formed from said droplets by passing them through a counter-current flow of cryogenic gas; b) freeze-drying the pellets; c) storing and homogenizing the freeze-dried pellets; d) assaying the freeze-dried pellets while they are being stored and homogenized; and e) loading the freeze-dried pellets into said containers.

The liquid pharmaceutical formulations according to the invention are suitable for parenteral administration. Parenteral administration includes, inter alia, intravenous injection or infusion, intra-arterial injection or infusion (into an artery), intra-muscular injection, intra-thecal injection, subcutaneous injection, intra-peritoneal injection or infusion, intra-osseous administration or injection into a tissue. The compositions according to the invention are particularly suitable for subcutaneous administration. Administration forms suitable for parenteral administration are inter alia preparations for injection or infusion in the form of solutions, suspensions, emulsions, in liquid form, or as lyophilizates or sterile powders, which are reconstituted before administration. If desired, the liquid pharmaceutical formulations according to the invention may also be freeze-dried and reconstituted before administration while maintaining the biological activity. However, freeze-drying of the antibody formulation according to the invention by conventional methods leads to lyophilizates with reconstitution times of up to two hours and more. Such long reconstitution times are cumbersome and impracticable as well for healthcare practitioners as for patients. Therefore, a spray-freezing-based method for the production of freeze-dried pellets comprising anti-FXIa antibodies which exhibit a significantly reduced reconstitution time as compared to FXIa antibody comprising lyophilizates obtained by conventional freeze-drying has been developed. This spray-freezing-based method, as described herein (Method 3), leads to freeze-dried anti-FXIa antibody comprising pellets with a reconstitution time of approximately 10 minutes when reconstituted to an antibody concentration of about 150 mg/ml. The spray-freeze-drying method for reducing the reconstitution time of freeze-dried pellets comprising an anti-FXIa antibody as described herein (Method 3) and applied in example 7 of the present application comprises the steps of:

a) freezing droplets of a solution comprising an anti-FXIa antibody to form pellets;

b) freeze-drying the pellets;

wherein in step a) the droplets are formed by means of droplet formation of the solution comprising an anti-FXIa antibody into a cooling tower which has a temperature-controllable inner wall surface and an interior temperature below the freezing temperature of the solution and in step b) the pellets are freeze-dried in a rotating receptacle which is housed inside a vacuum chamber.

Creation of frozen pellets for Method 3 can be performed according to any known technology. Importantly, however, dropping antibody comprising droplets into liquid nitrogen to therein form pellets is to be avoided.

In view of the subsequent freeze-drying step of Method 3, the frozen pellets favorably have a narrow particle size distribution. Afterwards the frozen pellets can be transported under sterile and cold conditions to a freeze dryer. The pellets are then distributed across the carrying surfaces inside the drying chamber by the rotation of the receptacle. Sublimation drying is in principle possible in any kind of freeze dryers suited for pellets. Freeze dryers providing space for sublimation vapor flow, controlled wall temperatures and suitable cross sectional areas between drying chamber and condenser are preferred.

The droplets used in step a) of Method 3 can be formed by means of droplet formation of the solution by passing through frequency-assisted nozzles. Preferably the oscillating frequency is ≥200 Hz to ≤5000 Hz, more particularly ≥400 Hz to ≤4000 Hz or ≥1000 Hz to ≤2000 Hz.

Independent of the nozzle being frequency-assisted, the diameter of the nozzle opening can be in the range of from 100 μm to 500 μm, preferably in the range of from 200 μm to 400 μm, very preferably in the range of from 300 μm to 400 μm. Said nozzle diameters result in droplet sizes in the range from about 200 μm to about 1000 μm, preferably in the range of from about 400 μm to about 900 μm, very preferably in the range of from about 600 μm to 800 μm.

In this context a size of “about” a given value, e.g. the upper or lower limit of a given size range, is to be understood as encompassing all droplet sizes deviating up to ±30% from this given value. For example a resulting droplet size of about 400 μm encompasses droplet sizes varying between 280 μm and 520 μm. Similarly, the size range of from about 100 μm to about 500 μm is to be understood as encompassing droplet sizes from 70 mm to 650 μm.

The droplets display a certain droplet size distribution around a median value which should be about the one referenced to above.

The pellet size median of the pellets obtained in step a) of the method described above is about ≥200 μm to about ≤1500 μm. Preferred is a pellet size median of about ≥500 μm to about ≤900 μm.

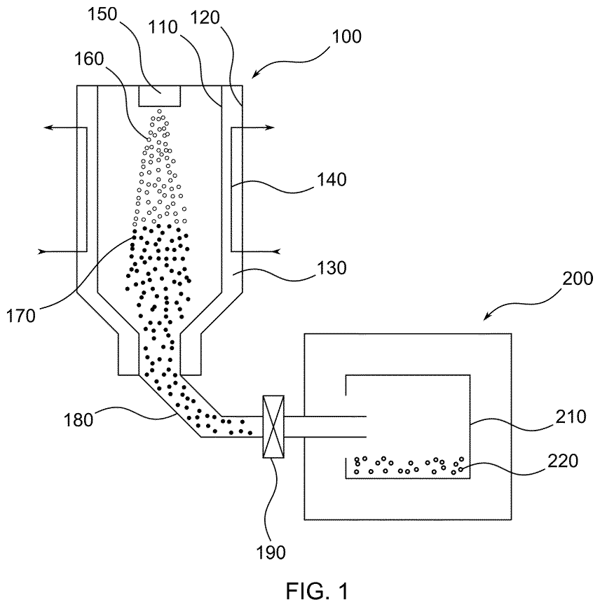

FIG. 1 schematically depicts an apparatus for conducting the spray-freeze-drying-based method for reducing the reconstitution time of freeze-dried pellets comprising an anti-FXIa antibody, as described above. The apparatus comprises, as main components, the cooling tower 100 and the vacuum drying chamber 200. The cooling tower comprises an inner wall 110 and an outer wall 120, thereby defining a space 130 between the inner wall 110 and the outer wall 120. This space 130 houses a cooling means 140 in the form of piping. A coolant can enter and leave the cooling means 140 as indicated by the arrows of the drawing. Coolant flowing through the cooling means 140 leads to a cooling of the inner wall 110 and thus to a cooling of the interior of the cooling tower 100. In the production of frozen pellets (cryopellets), liquid is sprayed into the cooling tower via nozzle 150. Liquid droplets are symbolized in accordance with reference numeral 160. The liquid droplets eventually solidify (freeze) on their downward path, which is symbolized in accordance with reference numeral 170. Frozen pellets 170 travel down a chute 180 where a valve 190 permits entry into the vacuum drying chamber 200. While not depicted here, it is of course also possible and even preferred that the chute 180 is temperature-controlled in such a way as to keep the pellets 170 in a frozen state while they are collecting before the closed valve 190. Inside the vacuum drying chamber 200 a rotatable drum 210 is located to accommodate the frozen pellets to be dried. The rotation occurs around the horizontal axis in order to achieve an efficient energy transfer into the pellets. Heat can be introduced through the drum or via an encapsulated infrared heater. As an end result, freeze-dried pellets symbolized by the reference numeral 220 are obtained.

The inner surface of the cooling tower used in the method described above has a temperature of not warmer than −120° C., preferably ≥−180° C. to ≤−120° C. Preferably the temperature is ≥−160° C. to ≤−140° C.

The above referred to temperatures of ≥−160° C. to ≤−140° C. are optimized for droplet sizes in the range of about ≥600 μm to about ≤800 μm that are frozen while falling a distance of 2 m to 4 m, particularly about 3 m.

The inner surface of the cooling tower is cooled by passing a coolant through one or more pipes which are in thermal contact with the inner surface. The coolant may be liquid nitrogen or nitrogen vapor of a desired temperature.

When using the apparatus as depicted in FIG. 1, the spray-freeze-drying based method for reducing the reconstitution time of freeze-dried pellets comprising an anti-FXIa antibody as described herein (Method 3) comprises the steps of:

a) freezing droplets of a solution comprising an anti-FXIa antibody to form pellets;

b) freeze-drying the pellets;

wherein in step a) the droplets are formed by means of droplet formation of the solution comprising an anti-FXIa antibody into a cooling tower (100) which has a temperature-controllable inner wall surface (110) and an interior temperature below the freezing temperature of the solution and wherein in step b) the pellets are freeze-dried in a rotating receptacle (210) which is housed inside a vacuum chamber (200).

Furthermore, Method 3 further can comprise the steps c) and d) after step b):

c) storing and homogenizing the freeze-dried pellets

d) loading the freeze-dried pellets into containers.

The storing and homogenization step c) can also be performed in the rotating receptacle within the vacuum chamber used for freeze-drying. In step d) user defined amounts of freeze-dried pellets are filled into the final containers. The storage containers are transferred to an isolated filling line and docked at a sterile docking station. The contents of the containers are transferred inside the isolator to the storage of the filling machine. Method 3 which results in no or only minimal damage to the processed anti-FXIa antibody allows for precise filling of the desired antibody amount within narrow specified ranges. The method further allows for flexible and individualized filling into containers for final use.

In the context of the present invention, the terms “conventional freeze-drying” and “conventionally freeze-dried” refers to a standard freeze-drying process in vials carried out in a standard freeze-drying chamber comprising one or more trays or shelves within a (vacuum) drying chamber and does not include the process step of spray-freezing. Typically, the product to be freeze-dried is filled into vials which are then placed into the (vacuum) drying chamber.

In the context of the present invention, the term “reducing the reconstitution time of freeze-dried pellets as compared to lyophilizates obtained by conventional freeze-drying” is to be understood as a reduction of the time period required for the complete or near complete dissolution of the freeze-dried pellets obtained by the method according to the present invention upon addition of the reconstitution medium, e.g. sterile water, as compared to lyophilizates obtained by conventional freeze-drying. The reconstitution time is particularly reduced by at least 10%, at least 15%, at least 20%, at least 25%, at least 30%, at least 40%, at least 50%, at least 60%, at least 70%, at least 80%, at least 90% or at least 95%. In the context of the present invention, the term “complete or near complete reconstitution/dissolution of freeze-dried pellets” refers to dissolution of at least 98% of the solids content of the freeze-dried pellets in the reconstitution medium, more particularly of at least 98.5% of the solids content of the freeze-dried pellets, most particularly at least 99%, at least 99.5%, at least 99.75% or at least 99.9% of the solids content of the freeze-dried pellets.

The operating principle of Method 3 has several distinct advantages. Firstly, the sprayed droplets of the anti-FXIa antibody comprising solution do not contact a cryogenic gas in a counter-flow fashion such as described in WO2006/008006 A1. There is no need for introducing a cryogenic gas into the interior space of the cooling tower and hence all handling and sterilization steps for the cryogenic gas can be omitted. All steps of this method can be carried out under sterile conditions and without compromising sterility between the individual steps.

Secondly, this method (Method 3) was experimentally found not to result in significant damages to the anti-FXIa antibody, thus avoiding binding affinity losses in the final product. In fact, anti-FXIa antibody comprising freeze-dried pellets obtained by this method (Method 3) exhibited increased binding affinity towards the FXIa antigen as assessed by indirect ELISA compared to anti-FXIa antibody comprising lyophilizates obtained by conventional freeze-drying (Method 1) or the freeze-drying process according to WO2006/008006 (Method 2). The avoidance of damages to the anti-FXIa antibody allows precise filling of a desired amount of active anti-FXIa antibody within a narrow specified range. Furthermore, this method allows for more flexibility in filing of the freeze-dried pellets in diverse volumes and application systems as compared to conventional lyophilization.

Thirdly, by conducting the freeze-drying step in a rotating receptacle inside the vacuum chamber the spatial position of each individual pellet is evenly distributed over time. This ensures uniform drying conditions and therefore eliminates spatial variations of antibody activity, e.g., binding affinity, as would be the case for freeze-dried vials on a shelf.

Last, it was found that anti-FXIa antibody comprising pellets produced according to this method (Method 3) exhibit a considerably shortened reconstitution time in particular as compared to anti-FXIa antibody comprising lyophilisates obtained by conventional freeze-drying (Method 1) but also as compared to pellets obtained by the process disclosed in WO2006/008006 A1 (Method 2).

However, in contrast to many other liquid antibody formulations, which are not stable over longer time periods, the liquid high-concentration antibody formulations according to the invention surprisingly exhibit high stability in long term tests which renders lyophilization with all its disadvantages and limitations usually unnecessary.

As used herein, “stable” formulations of biologically active proteins are formulations that exhibit reduced aggregation and/or reduced loss of biological activity of at least 20% upon storage at 2-8° C. for at least 6 month or upon storage of at least 12 month at <−60° C. compared with a control formula sample. Or alternatively which exhibit reduced aggregation and/or reduced loss of biological activity under conditions of thermal stress, e.g. multiple freeze/thaw cycles or agitation stress, e.g. (300 rpm for 3 h) etc.

The liquid pharmaceutical formulations according to the invention have valuable pharmacological properties and can be used for prevention and treatment of diseases in humans and animals. The liquid pharmaceutical formulations according to the invention which may be employed for diseases and treatment thereof particularly include the group of thrombotic or thromboembolic diseases. Accordingly, the liquid pharmaceutical formulations according to the invention are suitable for the treatment and/or prophylaxis of diseases or complications which may arise from the formation of clots.

In the context of the present invention, the “thrombotic or thromboembolic diseases” include diseases which occur both in the arterial and in the venous vasculature and which can be treated with the liquid pharmaceutical formulations according to the invention, in particular diseases in the coronary arteries of the heart, such as acute coronary syndrome (ACS), myocardial infarction with ST segment elevation (STEMI) and without ST segment elevation (non-STEMI), stable angina pectoris, unstable angina pectoris, reocclusions and restenoses after coronary interventions such as angioplasty, stent implantation or aortocoronary bypass, but also thrombotic or thromboembolic diseases in further vessels leading to peripheral arterial occlusive disorders, pulmonary embolisms, venous thromboembolisms, venous thromboses, in particular in deep leg veins and kidney veins, transitory ischaemic attacks and also thrombotic stroke and thromboembolic stroke.

Stimulation of the coagulation system may occur by various causes or associated disorders. In the context of surgical interventions, immobility, confinement to bed, infections, inflammation or cancer or cancer therapy, inter alia, the coagulation system can be highly activated, and there may be thrombotic complications, in particular venous thromboses. The liquid pharmaceutical formulations according to the invention are therefore suitable for the prophylaxis of thromboses in the context of surgical interventions in patients suffering from cancer. The liquid pharmaceutical formulations according to the invention are therefore also suitable for the prophylaxis of thromboses in patients having an activated coagulation system, for example in the stimulation situations described.

The liquid pharmaceutical formulations according to the invention are therefore also suitable for the prevention and treatment of cardiogenic thromboembolisms, for example brain ischaemias, stroke and systemic thromboembolisms and ischaemias, in patients with acute, intermittent or persistent cardiac arrhythmias, for example atrial fibrillation, and in patients undergoing cardioversion, and also in patients with heart valve disorders or with artificial heart valves.

In addition, the liquid pharmaceutical formulations according to the invention are suitable for the treatment and prevention of disseminated intravascular coagulation (DIC) which may occur in connection with sepsis inter alia, but also owing to surgical interventions, neoplastic disorders, burns or other injuries and may lead to severe organ damage through microthromboses.

Thromboembolic complications furthermore occur in microangiopathic haemolytical anaemias and by the blood coming into contact with foreign surfaces in the context of extracorporeal circulation, for example haemodialysis, ECMO (“extracorporeal membrane oxygenation”), LVAD (“left ventricular assist device”) and similar methods, AV fistulas, vascular and heart valve prostheses.

Moreover, the liquid pharmaceutical formulations according to the invention are suitable for the treatment and/or prophylaxis of diseases involving microclot formations or fibrin deposits in cerebral blood vessels which may lead to dementia disorders such as vascular dementia or Alzheimer's disease. Here, the clot may contribute to the disorder both via occlusions and by binding further disease-relevant factors.

Moreover, the liquid pharmaceutical formulations according to the invention can be used for inhibiting tumour growth and the formation of metastases, and also for the prophylaxis and/or treatment of thromboembolic complications, for example venous thromboembolisms, for tumour patients, in particular those undergoing major surgical interventions or chemo- or radiotherapy.

In addition, the liquid pharmaceutical formulations according to the invention can be used for the treatment or for prophylaxis of inflammatory diseases like rheumatoid arthritis (RA), or like neurological diseases like Alzheimer's disease (AD). Further on, these antibodies could be useful for the treatment of cancer and metastasis, thrombotic microangiopathy (TMA), age related macular degeneration, diabetic retinopathies, diabetic nephropathies, as well as other microvascular diseases.

Moreover, the liquid pharmaceutical formulations according to the invention can be used for the treatment and/or prophylaxis of Dialysis patients, especially the Cimino-fistula prevention of shunt thrombosis in hemodialysis. Hemodialysis can be performed using native arteriovenous fistulae, synthetic loop grafts, large-bore central venous catheters or other devices consisting of artificial surfaces. Administration of antibodies of this invention will prevent the formation of clot within the fistula (and propagation of embolized clot in the pulmonary arteries), both during dialysis and shortly thereafter.

Furthermore, the liquid pharmaceutical formulations according to the invention are also useful for the treatment and/or prophylaxis of patients undergoing intracardiac and intrapulmonary thromboses after cardiopulmonary bypass surgeries (e.g. ECMO: Extra-corporeal membrane oxygenation).

There is a high need for anticoagulation in dialysis patients without increasing the risk of unwanted bleeding events and where the incidence of venous thromboembolism (VTE) and atrial fibrillation (e.g. end-stage renal disease in hemodialysis patients) in this population is high. The liquid pharmaceutical formulations according to the invention are also useful for the treatment and/or prophylaxis of these types of patients.

The liquid pharmaceutical formulations according to the invention are also useful for the treatment and/or prophylaxis of patients affected with idiopathic thrombocytopenic purpura (IPT). These patients have an increased thrombotic risk compared to the general population. The concentration of the coagulation factor FXI is significantly higher in ITP patients compared to controls and aPTT is significantly longer in ITP patients.

In addition, the liquid pharmaceutical formulations according to the invention are also suitable for the prophylaxis and/or treatment of pulmonary hypertension.

In the context of the present invention, the term “pulmonary hypertension” includes pulmonary arterial hypertension, pulmonary hypertension associated with disorders of the left heart, pulmonary hypertension associated with pulmonary disorders and/or hypoxia and pulmonary hypertension owing to chronic thromboembolisms (CTEPH).

“Pulmonary arterial hypertension” includes idiopathic pulmonary arterial hypertension (IPAH, formerly also referred to as primary pulmonary hypertension), familial pulmonary arterial hypertension (FPAH) and associated pulmonary arterial hypertension (APAH), which is associated with collagenoses, congenital systemic-pulmonary shunt vitia, portal hypertension, HIV infections, the ingestion of certain drugs and medicaments, with other disorders (thyroid disorders, glycogen storage disorders, Morbus Gaucher, hereditary teleangiectasia, haemoglobinopathies, myeloproliferative disorders, splenectomy), with disorders having a significant venous/capillary contribution, such as pulmonary-venoocclusive disorder and pulmonary-capillary haemangiomatosis, and also persisting pulmonary hypertension of neonatants.

Pulmonary hypertension associated with disorders of the left heart includes a diseased left atrium or ventricle and mitral or aorta valve defects.

Pulmonary hypertension associated with pulmonary disorders and/or hypoxia includes chronic obstructive pulmonary disorders, interstitial pulmonary disorder, sleep apnoea syndrome, alveolar hypoventilation, chronic high-altitude sickness and inherent defects.

Pulmonary hypertension owing to chronic thromboembolisms (CTEPH) comprises the thromboembolic occlusion of proximal pulmonary arteries, the thromboembolic occlusion of distal pulmonary arteries and non-thrombotic pulmonary embolisms (tumour, parasites, foreign bodies).

The present invention further provides for the use of the liquid pharmaceutical formulations according to the invention for production of medicaments for the treatment and/or prophylaxis of pulmonary hypertension associated with sarcoidosis, histiocytosis X and lymphangiomatosis.

In addition, the liquid pharmaceutical formulations according to the invention are also suitable for the treatment and/or prophylaxis of disseminated intravascular coagulation in the context of an infectious disease, and/or of systemic inflammatory syndrome (SIRS), septic organ dysfunction, septic organ failure and multiorgan failure, acute respiratory distress syndrome (ARDS), acute lung injury (ALI), septic shock and/or septic organ failure.

In the course of an infection, there may be a generalized activation of the coagulation system (disseminated intravascular coagulation or consumption coagulopathy, herein below referred to as “DIC”) with microthrombosis in various organs and secondary haemorrhagic complications. Moreover, there may be endothelial damage with increased permeability of the vessels and diffusion of fluid and proteins into the extravasal space. As the infection progresses, there may be failure of an organ (for example kidney failure, liver failure, respiratory failure, central-nervous deficits and cardiovascular failure) or multiorgan failure.

In the case of DIC, there is a massive activation of the coagulation system at the surface of damaged endothelial cells, the surfaces of foreign bodies or crosslinked extravascular tissue. As a consequence, there is coagulation in small vessels of various organs with hypoxia and subsequent organ dysfunction. A secondary effect is the consumption of coagulation factors (for example factor X, prothrombin and fibrinogen) and platelets, which reduces the coagulability of the blood and may result in heavy bleeding.

The liquid pharmaceutical formulations according to the invention are also suitable for the primary prophylaxis of thrombotic or thromboembolic disorders and/or inflammatory disorders and/or disorders with increased vascular permeability in patients in which gene mutations lead to enhanced activity of the enzymes, or increased levels of the zymogens and these are established by relevant tests/measurements of the enzyme activity or zymogen concentrations.

The present invention further provides for the use of the liquid pharmaceutical formulations according to the invention for the treatment and/or prophylaxis of disorders, especially the disorders mentioned above.

The present invention further provides for the use of the liquid pharmaceutical formulations according to the invention for production of a medicament for the treatment and/or prophylaxis of disorders, especially the disorders mentioned above.

The present invention further provides a method for treatment and/or prophylaxis of disorders, especially the disorders mentioned above, using a therapeutically effective amount of an inventive compound.

The present invention further provides the liquid pharmaceutical formulations according to the invention for use in a method for the treatment and/or prophylaxis of disorders, especially the disorders mentioned above, using a therapeutically effective amount of a compound according to the invention.

These well described diseases in humans can also occur with a comparable aetiology in other mammals and can be treated there with the liquid pharmaceutical formulations of the present invention.

In the context of this invention, the term “treatment” or “treat” is used in the conventional sense and means attending to, caring for and nursing a patient with the aim of combating, reducing, attenuating or alleviating a disease or health abnormality, and improving the living conditions impaired by this disease.

The present invention therefore further provides for the use of the liquid pharmaceutical formulations according to the invention for the treatment and/or prevention of disorders, especially the disorders mentioned above.

The present invention further provides for the use of the liquid pharmaceutical formulations according to the invention for production of a medicament for the treatment and/or prevention of disorders, especially the disorders mentioned above.

The present invention further provides for the use of the liquid pharmaceutical formulations according to the invention in a method for treatment and/or prevention of disorders, especially of the aforementioned disorders.

The present invention further provides a method for treating and/or preventing diseases, more particularly the aforementioned diseases, using an effective amount of one of the liquid pharmaceutical formulations according to the invention.

In a preferred embodiment, the treatment and/or prevention is parenteral administration of the liquid pharmaceutical formulations according to the invention. Particular preference is given to subcutaneous administration.

The pharmaceutical formulations according to the invention can be used alone or, if required, in combination with one or more other pharmacologically active substances, provided that this combination does not lead to undesirable and unacceptable side effects. The present invention therefore further provides medicaments comprising at least one of the compositions according to the invention and one or more further active ingredients, especially for the treatment and/or prevention of the aforementioned diseases.

The liquids according to the invention can be administered as a single treatment but can also be administered repeatedly successively, or can be administered long-term following diagnosis.

### Example 1: Influence of Antibody Concentration on Viscosity and Particle Formation

PCT/EP2018/050951 describes a low concentration formulation of anti-FXIa antibody 076D-M007-H04-CDRL3-N110D comprising 25 mg/ml 076D-M007-H04-CDRL3-N110D in 10 mM L-histidine, 130 mM glycine, 5% trehalose dihydrate, 0.05% polysorbate 80 at pH 6.0 which is especially suitable for intravenous administration.

For a subcutaneous application it is important to determine the concentration maximum of the composition which, when concentrated in this particular formulation, resulted in increased viscosity values and particle formation. Clinical scenarios of the needed dose proposed a concentration target of approximately 150 mg/ml. Therefore, the concentration of 076D-M007-H04-CDRL3-N110D was increased using a centrifuge (Sigma, Typ 3K30) at 2000 G in combination with a centrifugation-tube (Merck Milipore, Amicon Ultra-15) containing a 30 kDa filter membrane that separated the composition and the antibody.

076D-M007-H04-CDRL3-N110D was formulated at increasing concentrations in the histidine/glycine buffer system as described in PCT/EP2018/050951 for the low-concentration formulation of 076D-M007-H04-CDRL3-N110D:
(1) 10 mM L-histidine, 130 mM glycine, 5% trehalose dihydrate, 0.05% polysorbate 80 at pH 6.0

Compositions in this examples as well as compositions in the examples below were analyzed regarding antibody concentration using UV/VIS spectrometer (NanoDrop 2000, ThermoFisher Scientific) absorbing the wavelength at 280 nm. For possible light scattering, the test was also corrected at 320 nm.

Dynamic viscosity of the solution was measured using a small sample viscometer (mVroc, RheoSense). 250 μL of 076D-M007-H04-CDRL3-N110D samples were injected at flow rates of 50 μl/min to 100 μl/min though the flow channel at 20° C.

The particle formation was monitored using Flow cytometry (MFI, ProteinSimple, 2 μm-100 μm) and light obscuration (Pamas SVSS, Pamas) covering a range from 2 μm to 100 μm of particle size.

TABLE 1
Influence of the concentration of 076D-M007-H04-CDRL3-N110D
on viscosity and particle formation in composition 1

| Antibody | | |
|---|---|---|
| Concentration | Dynamic viscosity | Particle count 2 μm-100 μm |
| mg/ml | mPa*s | Particles /ml |
| 10 | 1.14 | 1097 |
| 20 | 1.40 | 3004 |
| 40 | 2.25 | 5767 |
| 60 | 3.97 | 6681 |
| 80 | 6.85 | 8327 |
| 100 | 13.30 | 9833 |
| 110 | 18.57 | 10000 |
| 120 | 30.73 | 17497 |
| 130 | 58.29 | 10462 |
| 139.5 | 129.07 | 34146 |

Table 1 summarizes the viscosity and particle load measured with increasing 076D-M007-H04-CDRL3-N110D concentration in composition 1. It was not possible to increase the concentration of 076D-M007-H04-CDRL3-N110D up to the proposed range of approximately 150 mg/ml without increasing particle formation and exceeding the acceptable limits for viscosity at about 30 mPa*s. Therefore, the low concentration formulation for FXIa antibodies comprising a histidine/glycine buffer system (formulation 1) as described in PCT/EP2018/050951 was found not to be suitable for a high-concentration formulation of 076D-M007-H04-CDRL3-N110D as necessary for subcutaneous administration.

### Example 2: Influence of Different Excipients

To lower the viscosity and increase the antibody concentration the influence of different excipients was tested. This example shows the influence of different excipients on the attributes viscosity and second virial coefficient.

The second virial coefficient (B22 value) was determined by measuring the static light scattering (SLS) at 658 nm wavelength in dependence of the compositions antibody concentration in a range from 1 mg/ml to 10 mg/ml (NanoStar, Wyatt Technologies). By static light scattering, intermolecular interactions can be monitored. If the molecular masses increase disproportionately with increasing concentration, the antibodies tend to aggregation. The predominant conditions in the formulation are referred to as “attractive”. If, in contrast, the molecular masses decrease disproportionately, “repulsive” conditions prevail in the system. The tendency to aggregation is limited.

076D-M007-H04-CDRL3-N110D was formulated at approximately 120.0 mg/ml in a histidine-glycine buffer system comprising 10 mM L-Histidine and 130 mM Glycine at pH 6.0 (composition 2) with different excipients at concentrations of 50 mM, 75 mM and 150 mM respectively. The following compositions were tested:
(2) 10 mM L-Histidine, 130 mM Glycine pH at 6.0
(3) 10 mM L-Histidine, 130 mM Glycine, 50 mM sodium chloride at pH 6.0
(4) 10 mM L-Histidine, 130 mM Glycine, 75 mM sodium chloride at pH 6.0
(5) 10 mM L-Histidine, 130 mM Glycine, 150 mM sodium chloride at pH 6.0
(6) 10 mM L-Histidine, 130 mM Glycine, 50 mM calcium chloride-dihydrate at pH 6.0
(7) 10 mM L-Histidine, 130 mM Glycine, 75 mM calcium chloride-dihydrate at pH 6.0
(8) 10 mM L-Histidine, 130 mM Glycine, 150 mM calcium chloride-dihydrate at pH 6.0
(9) 10 mM L-Histidine, 130 mM Glycine, 50 mM L-Lysine hydrochloride at pH 6.0
(10) 10 mM L-Histidine, 130 mM Glycine, 75 mM L-Lysine hydrochloride at pH 6.0
(11) 10 mM L-Histidine, 130 mM Glycine, 150 mM L-Lysine hydrochloride at pH 6.0
(12) 10 mM L-Histidine, 130 mM Glycine, 50 mM L-Arginine hydrochloride at pH 6.0
(13) 10 mM L-Histidine, 130 mM Glycine, 75 mM L-Arginine hydrochloride at pH 6.0
(14) 10 mM L-Histidine, 130 mM Glycine, 150 mM L-Arginine hydrochloride at pH 6.0

TABLE 2
Influence of different excipients on viscosity and second
virial coefficient (B22)

| Composition | Dynamic viscosity | Second virial coefficient |
|---|---|---|
| No. | mPa*s | ml * mol/g² |
| 2 | 39.8 | −3.36E−05 |
| 3 | 23.1 | 2.42E−05 |
| 4 | 17.9 | 1.02E−05 |
| 5 | 12.7 | 9.56E−06 |
| 6 | 9.49 | 1.30E−05 |
| 7 | 8.26 | 1.76E−05 |
| 8 | 7.60 | 1.31E−04 |
| 9 | 14.3 | 6.49E−05 |
| 10 | 13.7 | 2.20E−05 |
| 11 | 10.7 | 3.07E−05 |
| 12 | 8.99 | 3.95E−05 |
| 13 | 7.20 | 5.59E−05 |
| 14 | 7.31 | 7.05E−05 |

Table 2 summarizes the dynamic viscosity and the second virial coefficient for compositions 2 to 14. Viscosity values decreased from 39.8 mPa*s (for composition 2 comprising a histidine-glycine-buffer system without further excipients) with all of the tested excipients up to five-fold. In general the viscosity-lowering effect increased with increasing amounts of the excipient. Sodium chloride, lysine, calcium chloride and arginine lowered the viscosity of the solution at a concentration of 150 mM to 12.7 mPa*s, 10.7 mPa*s, 7.6 mPa*s and 7.31 mPa*s respectively. However, the lowest viscosity was achieved using 75 mM arginine with a resulting viscosity of 7.2 mPa*s.

Arginine was selected as it was the most effective excipient to reduce the viscosity in this system. Further experiments were conducted using arginine as viscosity reducing agent. However, further investigations to balance the reduced viscosity with antibody stability were still needed.

Furthermore, the dynamic viscosity values (Table 2) of composition (14) indicated a significant change of protein-protein interaction. Therefore, compositions (2) and (14) were exemplary tested under stress conditions.

As arginine was the most effective viscosity reducing agent and also showed a positive effect on the second virial coefficient, composition 14 (containing 150 mM arginine) was tested by provoking particle generation under different stress conditions in comparison to starting composition 2 (without excipient). Three different stress conditions which may potentially lead to aggregation of the protein and formation of oligomers (HMW) up to visible particles were induced to compositions 2 and 14. Tested stress conditions were agitation stress (300 rpm for 3 h) using a shaker (Type HS 260C, IKA), 3 Freeze/thaw cycles from −20° C. to 20° C. for 6 hours each and storage at 2-8° C. for 1 week.

TABLE 3
Stability of compositions 2 and 14 after different stress conditions

| | Storing | | |
|---|---|---|---|
| | Test | Freeze/thaw Test | Agitation |
| Compo- | particle count | particle count | Test particle |
| sition | 2 μm-100 μm | 2 μm-100 gm | formation |
| No. | Particles/ml | Particles/ml | Particles/ml |
| 2 | 4610 | 6938 | 32773 |
| 14 | 813 | 2969 | 23767 |

As shown in Table 3, addition of arginine (composition 14) had an overall positive effect on the particle forming behaviour of 076D-M007-H04-CDRL3-N110D under all three stress conditions in comparison to composition 2 without a viscosity reducing excipient.

### Example 3: Influence of pH

Besides different excipients a change of pH can influence the viscosity and stability of an antibody. A pH range from pH 4.7 to 7.4 is regarded as suitable for subcutaneous application.

Second virial coefficient and particle formation were assessed as described earlier. The thermal stability of the compositions was determined by measuring the fluorescence of intrinsic and extrinsic tryptophan sources in the antibody containing compositions. The compositions were heated in a temperature profile from 15° C. to 95° C. using a differential scanning fluorimetry (DSF) method (Prometheus, NanoTemper) and collecting fluorescence data at 330 nm and 350 nm wavelength. An increased melting temperature (Tₘ) measured with DSF is a strong indication for increased conformational stability.

076D-M007-H04-CDRL3-N110D was formulated at approximately 120 mg/ml in 10 mM L-Histidine, 130 mM Glycine and 75 mM L-Arginine hydrochloride at three different pH-values. Following compositions were tested:
(15) 10 mM L-Histidine, 130 mM Glycine, 75 mM L-Arginine hydrochloride, pH 6.0
(16) 10 mM L-Histidine, 130 mM Glycine, 75 mM L-Arginine hydrochloride, pH 5.5
(17) 10 mM L-Histidine, 130 mM Glycine, 75 mM L-Arginine hydrochloride, pH 5.0

TABLE 4
Influence of pH steps on second virial coefficient, particle
formation and thermal stability of compositions 15-17

| Compo- | Second virial | Particle count | Thermal |
|---|---|---|---|
| sition | coefficient | 2 μm-100 μm | stability |
| No. | ml * mol/g² | Particles/ml | ° C. |
| 15 | 7.43E−06 | 294 | 66.38 |
| 16 | 1.15E−05 | 28 | 64.71 |
| 17 | 1.14E−04 | 31 | 59.36 |

Table 4 summarizes second virial coefficient, particle formation as well as thermal stability of compositions 15 to 17. Lowering the pH from pH 6.0 to pH 5.5 and pH 5.0 respectively increased the second virial coefficient from 7.43E-06 ml*mol/g² to 1.14E-04 ml*mol/g² and decreased the particle formation that was induced due to the sample processing from about 294 particles >2 μm to 31 particles. Tₘ values however decreased from 66.38° C. to 59.36° C.

Due to the positive effect onto the second virial coefficient it was decided that a reduced pH at approximately 5.0 was preferred for further experiments. However, the trade of with decreased conformational stability was noted and further addressed in Example 5.

### Example 4: Influence of Surfactant Concentration

This example shows the effect of increasing surfactant concentrations on the compositions stability in terms of sub visible particle formation using Micro Flow Imaging (MFI 5200, Protein Simple) in a particle range from 2 μm to 100 μm. The compositions were exposed to different stress conditions as described in Example 2. The selected surfactant was polysorbate 80.

076D-M007-H04-CDRL3-N110D was formulated at approximately 150 mg/ml in 20 mM L-Histidine at pH 5.0 with increasing concentrations of polysorbate 80. The following compositions were tested:
(18) 20 mM L-Histidine, pH 5.0, 0.00% polysorbate 80
(19) 20 mM L-Histidine, pH 5.0, 0.01% polysorbate 80
(20) 20 mM L-Histidine, pH 5.0, 0.05% polysorbate 80
(21) 20 mM L-Histidine, pH 5.0, 0.10% polysorbate 80
(22) 20 mM L-Histidine, pH 5.0, 0.15% polysorbate 80
(23) 20 mM L-Histidine, pH 5.0, 0.20% polysorbate 80

TABLE 5
Influence of different surfactants concentrations on particle
formation of 076D-M007-H04-CDRL3-N110D - Agitation
stress induced in composition 18-23

| | | Particle | Particle | Particle |
|---|---|---|---|---|
| | Particle count | count | count | count |
| Compo- | 2 μm- | 5 μm- | 10 μm- | 25 μm- |
| sition | 100 μm | 100 μm | 100 μm | 100 μm |
| No. | Particles/ml | Particles/ml | Particles/ml | Particles/ml |
| 18 | 59077 | 25417 | 3819 | 68 |
| 19 | 40888 | 9064 | 2588 | 265 |
| 20 | 16155 | 3620 | 925 | 74 |
| 21 | 21419 | 4434 | 1078 | 74 |
| 22 | 18942 | 4488 | 1200 | 130 |
| 23 | 18739 | 3949 | 1315 | 92 |

Table 5 summarizes the particle formation of 076D-M007-H04-CDRL3-N110D while inducing agitation stress to the compositions.

TABLE 6
Influence of different surfactants concentrations on particle
formation of 076D-M007-H04-CDRL3-N110D - Freeze/thaw
stress induced in composition 20-23

| | | Particle | Particle | Particle |
|---|---|---|---|---|
| | Particle count | count | count | count |
| Compo- | 2 μm- | 5 μm- | 10 μm- | 25 μm- |
| sition | 100 μm | 100 μm | 100 μm | 100 μm |
| No. | Particles/ml | Particles/ml | Particles/ml | Particles/ml |
| 20 | 5275 | 897 | 51 | 3 |
| 21 | 4475 | 637 | 36 | 3 |
| 22 | 6692 | 469 | 48 | 0 |
| 23 | 9103 | 1386 | 69 | 0 |

Table 6 summarizes the particle formation of 076D-M007-H04-CDRL3-N110D while inducing freeze/thaw stress to the compositions.

The protective effect of the surfactant reached a plateau at a concentration of approximately 0.05% polysorbate 80 in composition (20) to 0.20% polysorbate 80 in composition 23. To ensure the protective effect over shelf life and create a secure robustness corridor 0.1% polysorbate 80 was particularly preferred. Additionally the protective effect of 0.1% polysorbate 80 (composition 21) as shown in Table 6 resulted in 637 particles >5 μm compared to 897 particles >5 μm in composition 20 while inducing freeze/thaw stress which indicates that the polysorbate 80 concentration should be at least 0.1%.

Higher concentrations of polysorbate 80, as shown in Table 5 and Table 6, showed no significant improvement in protective effects.

### Example 5: Combination of Different Excipients

This example shows a combined approach of the previous examples. Its purpose was to optimize the effects that were described earlier to lower the viscosity and improve the stability of 076D-M007-H04-CDRL3-N110D while giving more detailed information about the concentration range of arginine To lower the osmolality of the compositions to physiological levels (240-400 mOsm/kg) it was necessary to reduce the overall concentrations of the excipients.

The purpose of the following screening was to optimize the viscosity lowering but also particle formation preventing properties of the high-concentration formulation for 076D-M007-H04-CDRL3-N110D while reducing the content of arginine to 50 mM and revealing a beneficial effect at lower concentrations.

Also synergetic effects of arginine in combination with glycine and methionine were investigated. Therefore buffering systems with different excipients and combinations thereof at various pH values were set up and evaluated regarding second virial coefficient, thermal stability and viscosity.

076D-M007-H04-CDRL3-N110D was formulated at approximately 150 mg/ml in different compositions:
(24) 20 mM L-Histidine
(25) 20 mM L-Histidine, 50 mM L-Arginine hydrochloride
(26) 20 mM L-Histidine, 30 mM L-Arginine hydrochloride
(27) 20 mM L-Histidine, 50 mM L-Arginine hydrochloride, 10 mM L-Methionine
(28) 20 mM L-Histidine, 130 mM Glycine
(29) 20 mM L-Histidine, 50 mM L-Arginine hydrochloride, 130 mM Glycine
(30) 20 mM Phosphate buffer
(31) 20 mM Acetate buffer

All compositions were tested in a pH range from 5.0 to 6.0 in steps of 0.2.

TABLE 7
Influence of pH changes on the second virial coefficient (B22) of 076D-
M007-H04-CDRL3-N110D in different compositions

| | pH 5.0 | pH 5.2 | pH 5.4 | pH 5.6 | pH 5.8 | pH 6.0 |
|---|---|---|---|---|---|---|
| Composition | ml* | ml* | ml* | ml* | ml* | ml* |
| No. | mol/g² | mol/g² | mol/g² | mol/g² | mol/g² | mol/g² |
| 24 | −6.70E−05 | −1.13E−04 | −1.13E−04 | −1.56E−04 | −1.64E−04 | −2.70E−04 |
| 25 | 1.49E−05 | 8.38E−06 | 7.47E−06 | 3.58E−06 | −3.71E−05 | −2.80E−05 |
| 26 | −3.75E−05 | −4.98E−05 | −6.24E−05 | −1.40E−04 | −1.25E−04 | — |
| 27 | −8.50E−05 | −8.91E−05 | −6.03E−05 | −6.72E−05 | −8.53E−05 | −1.62E−04 |
| 28 | 8.89E−05 | 6.54E−05 | −7.11E−05 | −1.61E−04 | −2.60E−04 | −4.83E−04 |
| 29 | 2.94E−05 | — | — | — | — | — |
| 30 | −1.17E−04 | −1.08E−04 | −1.09E−04 | −1.07E−04 | −9.51E−05 | −1.56E−04 |
| 31 | 9.01E−06 | −1.82E−05 | −4.79E−05 | −5.52E−05 | −8.37E−05 | −8.51E−05 |

Table 7 summarizes the second virial coefficient of different compositions comprising approximately 150 mg/ml 076D-M007-H04-CDRL3-N110D in dependence of the compositions pH. As already described in Example 3 decreased pH overall resulted in an increased second virial coefficient. B22 values (representing intermolecular interactions) were between −2.70E-04 mol*ml/g² and 2.94E-05 mol*ml/g². The compositions 25, 28, 29 and 31 had a second virial coefficient above zero at pH 5.2 and lower (see Table 7) which was preferable as high B22 values are an indication of a colloidal stability. In contrast composition 26 showed no significant improvement although containing 30 mM arginine. This observed effect led to the conclusion that a concentration of only 30 mM arginine was not sufficient for a feasible high-concentration formulation of 076D-M007-H04-CDRL3-N110D.

TABLE 8
Influence of pH changes on the thermal stability (Tₘ) of 076D-M007-H04-
CDRL3-N110D in different compositions

| Composition | pH 5.0 | pH 5.2 | pH 5.4 | pH 5.6 | pH 5.8 | pH 6.0 |
|---|---|---|---|---|---|---|
| No. | ° C. | ° C. | ° C. | ° C. | ° C. | ° C. |
| 24 | 60.7 | 62.7 | 63.6 | 65.7 | 66.3 | 67.5 |
| 25 | 60.5 | 61.2 | 63.0 | 63.8 | 65.6 | 66.0 |
| 26 | — | — | — | — | — | — |
| 27 | 59.7 | 61.0 | 61.8 | 63.3 | 64.9 | 66.0 |
| 28 | 64.7 | 65.7 | 65.1 | 67.3 | 67.8 | 68.7 |
| 29 | | — | — | — | — | — |
| 30 | 66.5 | 67.0 | 67.9 | 68.6 | 69.2 | 69.8 |
| 31 | 66.1 | 67.2 | 68.3 | 69.0 | 69.7 | 78.5 |

Table 8 summarizes the thermal stability of different compositions comprising approximately 150 mg/ml 076D-M007-H04-CDRL3-N110D in dependence of the compositions pH. The Tₘ values were between 59.7° C. and 78.5° C. Overall decreased pH resulted in decreased Tₘ values. Compositions that were stabilized using the amino acids, histidine, glycine, arginine and/or methionine in different combinations and concentrations (24 to 28) had a lower Tₘ value than the compositions containing phosphate or acetate buffer.

Surprisingly among the compositions containing amino acids, composition 28 showed a significant higher Tₘ value of +4° C. to +5° C. in comparison to compositions without glycine, leading to the conclusion that glycine had a stabilizing effect on the antibody.

The positive effect of arginine on the second virial coefficient as well the positive effect of glycine on the thermal stability of 076D-M007-H04-CDRL3-N110D led to the conclusion that a preferable composition should include both amino acids in addition to histidine.

The combination of the excipients, glycine and arginine, in addition to histidine in composition 32 (20 mM histidine, 50 mM arginine, 50 mM glycine, 5% trehalose dihydrate, 0.10% polysorbate 80, pH 5.0) confirmed a synergistic effect. The second virial coefficient of composition 32 was 3.385E-05 ml*mol/g² and was therewith in the range of compositions 25, 28 and 29 (as depicted in Table 7). The Tₘ of composition 32 was with 63.3° C. comparable to composition 28 comprising only glycine in addition to histidine. These data show that combination of glycine and arginine in addition to histidine, reduced protein-protein interaction (second virial coefficient) and at the same time increased thermal stability (Tₘ).

TABLE 9
Influence of pH changes on the dynamic viscosity of 076D-
M007-H04-CDRL3-N110D in different compositions

| Compo- | | |
|---|---|---|
| sition | pH 5.0 | pH 6.0 |
| No. | mPa * s | mPa * s |
| 25 | 54.3 | 74.6 |
| 27 | 45.7 | — |
| 29 | 25.6 | — |
| 30 | — | 50.6 |

Table 9 shows the dynamic viscosity of different compositions comprising approximately 150 mg/ml 076D-M007-H04-CDRL3-N110D at pH 5.0 and pH 6.0. Although the second virial coefficients of compositions 25, and 29 were in a comparable range the dynamic viscosity of composition 29 is the only formulation that led to an acceptable viscosity of 25.6 mPa*s at pH 5.0.

Osmolality was measured using a freeze-point osmometer and a three point calibration (50, 300, 2000 mOsm/kg—Osmomat 030, GonoTech, Berlin). Composition 29 led to an osmolality of approximately 324 mOsm/kg without containing further surfactants as polysorbate 80 or stabilizers as trehalose dihydrate. It was known that the addition of 5% of trehalose dihydrate leads to additional 145 mOsm/kg increasing the compositions osmolality value. The resulting theoretical osmolality of composition 29 in combination with 5% of trehalose dihydrate was therefore with 469 mOsm/kg expected to be hypertonic and outside the acceptable range of 240-400 mOsm/kg.

The amount of glycine in composition 29 was therefore reduced from 130 mM to 50 mM (leading to composition 32). This reduction resulted in an osmolality of 241 mOsm/kg. The combination with 5% of trehalose dihydrate as stabilizer would than arithmetically lead to an acceptable osmolality of 386 mOsm/kg.

This arithmetical value was confirmed by measurement of the osmolality of composition 32 which showed an osmolarity of 371 mOsm/kg.

### Example 6: Lyophilization by Conventional Freeze-Drying

This example shows the suitability of the liquid high-concentration composition comprising 076D-M007-H04-CDRL3-N110D and a histidine-glycine-arginine buffer system for conventional lyophilization. Trehalose was added as stabilizer.

076D-M007-H04-CDRL3-N110D was formulated at approximately 150 mg/ml in:
(32) 20 mM L-Histidine, 50 mM L-Arginine hydrochloride, 50 mM Glycine, 5% trehalose dihydrate, 0.10% polysorbate 80, pH 5.0

To develop a suitable lyophilization process it was essential to determine the collapse temperature that decided at which temperature the primary drying could be conducted. The collapse temperature was measured using a lyo-microscope (Lyostat 2, Biopharma) by freezing the composition to −50° C. before drawing vacuum (0.1 mbar) and heating the sample with a ramp of 1° C./minute to 20.0° C. While heating up the composition pictures were taken and analysed until a collapse of the tested system could be observed.

The collapse temperature of 076D-M007-H04-CDRL3-N110D was found to be −14.3° C. and is an essential parameter for selection of the following lyophilization cycle.

The liquid composition 32 comprising anti-FXIa antibody 076D-M007-H04-CDRL3-N110D was processed according to a conventional freeze-drying method (Method 1). The solution containing 150 mg/ml anti-FXIa antibody was filled into 10R type I glass vials and freeze-dried in a conventional vial freeze dryer. A total of 20 vials were filled with 2.25 ml solution per vial, semi-stoppered and loaded into a Virtis Genesis freeze dryer. The solution was frozen to −45° C., and primary drying was performed at +10° C., followed by a secondary drying step at 40° C. The complete freeze drying process required approx. 38 hours. The vials were stoppered within the freeze dryer and sealed directly after unloading.

The details of the lyophilization cycle according to a conventional freeze-drying method (Method 1) for composition 32 are summarized in Table 12.

TABLE 12
Lyophilization cycle of composition 32 (Method 1)

| | Time | Temp | Pressure |
|---|---|---|---|
| | [hh:mm] | [° C.] | [mbar] |
| Loading | 00:01 | 20.0 | 1000 |
| Freezing | 00:30 | −5.0 | 1000 |
| Freezing | 01:00 | −5.0 | 1000 |
| Freezing | 00:40 | −45.0 | 1000 |
| Freezing | 03:30 | −45.0 | 1000 |
| Evacuation | 00:01 | −45.0 | 0.100 |
| Primary | 01:00 | 10 | 0.1 |
| drying | | | |
| Primary | 19:00 | 10 | 0.1 |
| drying | | | |
| Secondary | 01:00 | 40 | 0.04 |
| drying | | | |
| Secondary | 10:00 | 40 | 0.04 |
| drying | | | |

| | | |
|---|---|---|
| Time | Loading | 00:01 |
| Summary | Freezing | 05:41 |
| | Primary | 20:00 |
| | drying | |
| | Secondary | 11:00 |
| | drying | |
| | Total | 36:42 |

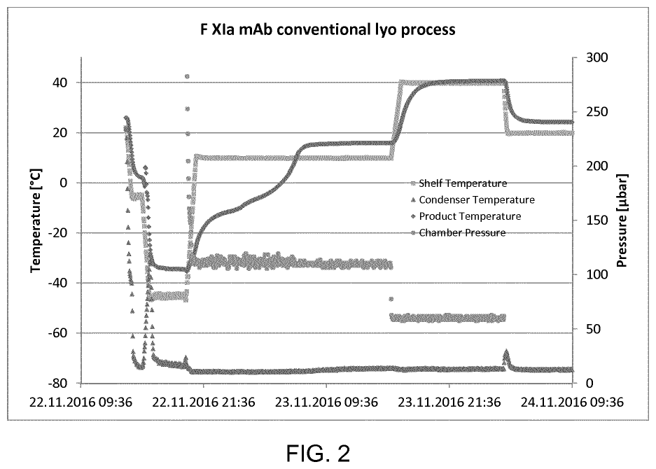

The pressure and temperature profile measured over time during the thus conducted conventional freeze-drying process is graphically depicted in FIG. 2.

The conventional lyophilization method described above resulted in a yellowish cake or powder. which can subsequently be reconstituted.

For reconstitution of the lyophilizate 2 ml sterile water for injection as reconstitution medium was injected into each of the vials. The vials were then gently agitated for about 10 to 20 seconds. Reconstitution of this lyophilizate obtained by conventional freeze-drying resulted in a reconstitution time of 137 min.

After reconstitution a clear, yellowish solution without any visible particles was observed. No aggregation or hints of aggregation were detected.

### Example 7: Lyophilization by Different Spray-Freeze-Drying Methodes

As the reconstitution time of the lyophilzate obtained by a conventional freeze-drying method as described in Example 6 (Method 1) was, with more than 2 hours, unacceptably long, two different other freeze-drying methods were applied and compared to the conventional freeze-drying as described above.

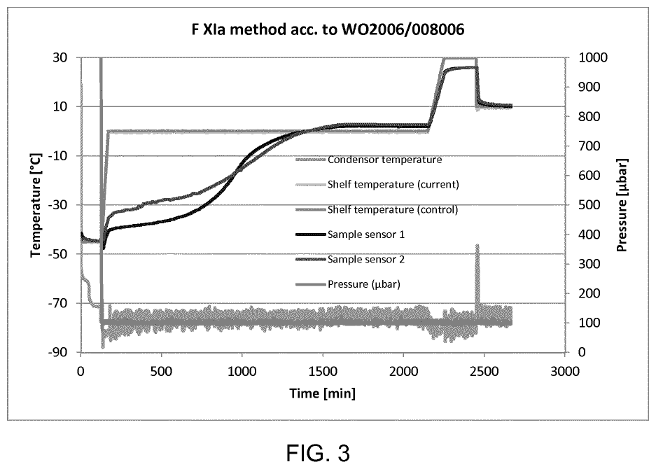

Firstly, the liquid composition 32 comprising anti-FXIa antibody 076D-M007-H04-CDRL3-N110D was processed according to the method described in WO 2006/008006 (Method 2). 138 ml solution containing 150 mg/ml anti-FXIa antibody were sprayed through a 400 μm nozzle and atomized at a frequency of 470 Hz with a rate of about 19.5 g/min and a pressure overlay of 220 mbar. The droplets were frozen in an isolated vessel filled with liquid nitrogen that was positioned approx. 25 cm below the nozzle and stirred throughout the process. After completion of spraying the frozen pellets were removed by pouring the liquid nitrogen through a pre-cooled sieve and placed in a steel rack lined with plastic foil onto the pre-cooled shelves of a Virtis Advantage Pro freeze dryer and lyophilized. Primary drying was conducted at 0° C. shelf temperature over a duration of 33 hours, followed by secondary drying for 5 hours at 30° C. After completion of drying, the dry pellets were instantly transferred into glass bottles which were firmly closed. Subsequently, 520 mg of pellets were weighed into 10R type I glass vials under a dry nitrogen atmosphere. The pressure and temperature profile measured over time during freezing and drying of the antibody solution according to the method described in WO 2006/008006 is graphically depicted in FIG. 3.

Secondly, the liquid composition 32 comprising anti-FXIa antibody 076D-M007-H04-CDRL3-N110D was processed according to the spray-freeze-drying based method for reducing the reconstitution time of freeze-dried pellets (“Method 3 as described herein) which comprises the steps of:

a) freezing droplets of a solution comprising an anti-FXIa antibody to form pellets;

b) freeze-drying the pellets;

wherein in step a) the droplets are formed by means of droplet formation of the solution comprising an anti-FXIa antibody into a cooling tower which has a temperature-controllable inner wall surface and an interior temperature below the freezing temperature of the solution and in step b) the pellets are freeze-dried in a rotating receptacle which is housed inside a vacuum chamber.

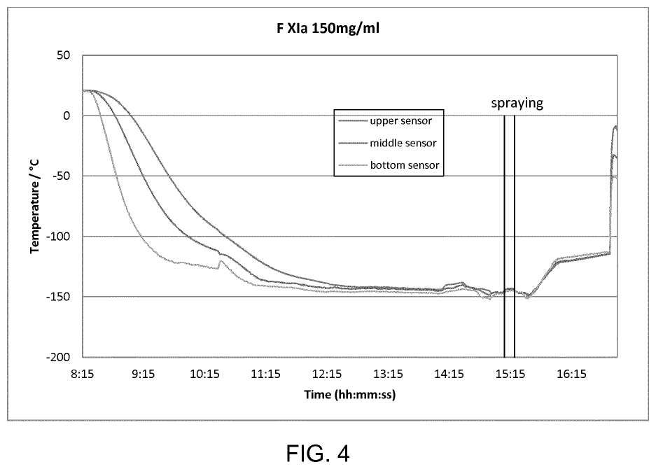

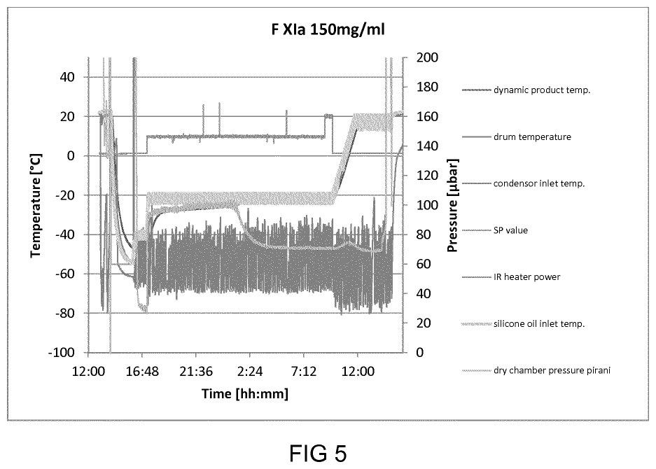

Therefore, 250 ml solution containing 150 mg/ml anti-FXIa antibody was freeze-dried by spraying the solution into a wall-cooled cooling tower. The spraying nozzle had one aperture with a diameter of 400 μm. This corresponds to a droplet size of about 800 μm. The oscillation frequency was 1445 Hz, the deflection pressure 0.4 bar and the pump was operated at 14 rpm. After completion of drying, the dry pellets were instantly transferred into glass bottles which were firmly closed. Subsequently, 520 mg of pellets were weighed into 1 OR type I glass vials under a dry nitrogen atmosphere. The temperature profile in the cooling tower measured over time is graphically depicted in FIG. 4. The temperature and pressure profile measured over time during freezing and drying of the antibody solution is graphically depicted in FIG. 5. Method 3 as described herein yielded uniform pellets exhibiting a narrow size and weight distribution and a high surface area. The residual humidity in the pellets obtained by this method was 0.268%.

The lyophilizates obtained by conventional freeze-drying (Method 1) comprised 0.15% residual moisture.

Size exclusion chromatography analyses of the pellets obtained by the three different freeze-drying processes are given in the Table 13.

TABLE 13
Size exclusion chromatography analyses of the pellets
obtained by the three different freeze-drying processes

| | SEC | | | |
|---|---|---|---|---|
| | | Sum high | Sum low | |
| | | molecular | molecular | |
| | Dimer | weight | weight | |
| | [% | (HMW) | (LMW) | Monomer |
| Sample | area] | aggregates | aggregates | [% Area] |
| Method 3 | 1.66 | 1.82 | 1.20 | 96.96 |
| (as described | | | | |
| herein) | | | | |
| Method 1 | 1.35 | 1.41 | 1.13 | 97.45 |
| (Conventional | | | | |
| Lyophilization) | | | | |
| Method 2 | 1.57 | 1.77 | 1.15 | 97.07 |
| (WO2006/008006) | | | | |

Overall, comparable analytical data were obtained by size exclusion chromatography for the three freeze-drying methods.

To determine the quantity of intact antibody relative to the overall proteinaceous components present in the sample, IgG purity was analyzed by Capillary SDS-Gel Electrophoresis (CGE). Test and reference samples were separated by CGE using a bare fused-silica capillary in the presence of sodium dodecyl sulfate (SDS). The test was performed under non-reducing conditions. The separated samples were monitored by absorbance at 220 nm. The intention of the assay was to integrate the peak area of the main peak and analyse the byproducts after reduction.

The results of capillary gel electrophoresis (CGE) and ELISA analyses are given in the Table 14.

TABLE 14
Capillary gel electrophoresis (CGE) and ELISA analyses
of the pellets obtained by the three different freeze-drying
processes

| CGE | | | | | |
|---|---|---|---|---|---|
| | IgG | HHL | HH | HL | |
| | [% corr. | [% corr. | [% corr. | [% corr. | |
| Sample | Area] | Area] | Area] | Area] | ELISA |
| Method 3 | 95.82 | 2.51 | 0.38 | 0.18 | 112 |
| | | | | | (111.95) |
| Method 1 | 95.80 | 2.60 | 0.36 | 0.17 | 101 |
| | | | | | (100.73) |
| Method 2 | 95.83 | 2.55 | 0.38 | 0.17 | 87 |
| | | | | | (86.87) |

Reconstitution times of the pellets obtained by the three different freeze-drying methods were compared as follows. 2 ml sterile water for injection as reconstitution medium was injected into each of the vials. After taking photographs the vials were gently agitated for about 10 to 20 seconds. Reconstitution of the pellets over time was visually observed and documented photographically.

The reconstitution times of the pellets obtained by the three different freeze-drying methods are given below:

| Freeze-Drying | Reconstitution | |
|---|---|---|
| method | Time | Ab Concentration |
| Method 1 | 137 min | 150 mg/ml |
| Method 2 | 16 min | 150 mg/ml |
| Method 3 | 11 min | 150 mg/ml |

The reconstitution of the freeze-dried anti-FXIa antibody comprising pellets obtained according to Method 3 as described herein was significantly faster than the reconstitution of equivalent anti-FXIa antibody comprising lyophilizates obtained by conventional freeze-drying (Method 1), but also faster compared to freeze-dried pellets obtained according to WO 2006/008006 (Method 2).

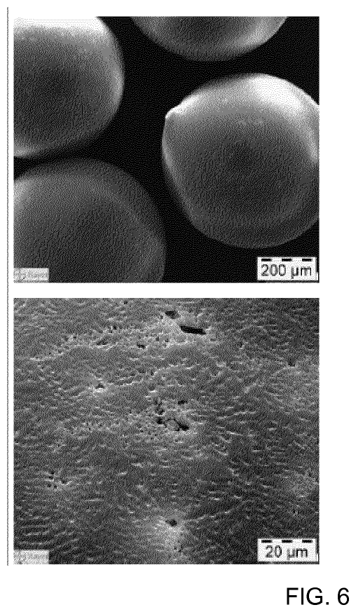

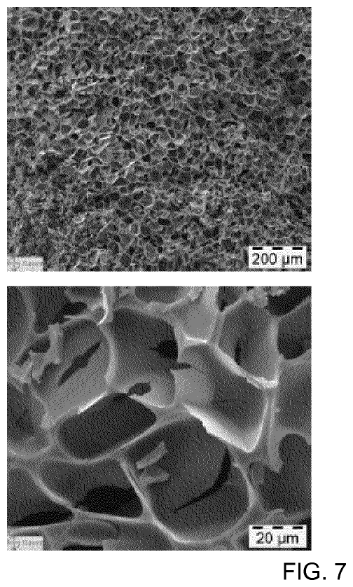

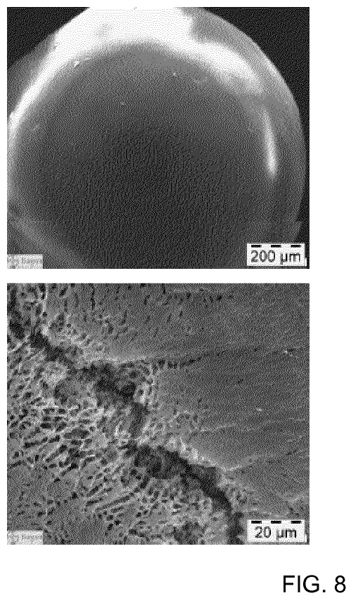

The pellets obtained by the three different freeze-drying methods were thereafter subjected to Scanning Electron Microscopy (SEM) measurements. Therefore, preparation of samples was performed in a glove bag under nitrogen atmosphere, each sample was prepared individually. The sample was placed on a holder and sputtered with gold. Subsequently the scanning electron microscopy measurement was performed. SEM pictures are shown in FIGS. 6 to 8.

It can be seen that the pellets produced pursuant to Method 3 as described herein display a particularly homogeneous morphology, which may improve handling properties in later process steps.

### Example 8: Long Term Stability of Lyophilized High-Concentration Formulation

This example describes the long term stability of the lyophilized high-concentration formulation of 076D-M007-H04-CDRL3-N110D at 2-8° C. and 25° C.

2.25 ml of composition 32 was filled in sterilized 6R glass vials. The liquid formulation was lyophilized according to the conventional freeze-drying method (Method 1) as described in example 6. The lyophilized composition 32 to be reconstituted to contain 150 mg/ml 076D-M007-H04-CDRL3-N110D comprised therefore 0.047 mg L-Histidine, 0.158 mg L-Arginine hydrochloride, 0.056 mg Glycine, 0.75 mg trehalose dihydrate, and 0.015 mg polysorbate 80 per mg of 076D-M007-H04-CDRL3-N110D.

Once reconstituted with water, the lyophilized composition had a pH of about 5.0.

The lyophilized composition was stored for a time period of 12 month at 2-8° C. and 25° C. At certain time points (3, 6, 9, 12, 18 and 24 months) samples were reconstituted with sterile water.

After reconstitution (final volume 2.25 ml/vial) the liquid compositions were analyzed for usability in pharmaceutical applications. In addition to the analytical methods described above which measures the physical stability (concentration, aggregation, particle formation, dynamic viscosity, osmolality, etc.) chemical stability as well as the activity of 076D-M007-H04-CDRL3-N110D were analysed.

The monomeric content was measured using size exclusion chromatography (SEC) that separated monomers from fragments (low molecular weight, LMW) and oligomers (high molecular weight, HMW) based on their spatial size. The separation of the fractions was achieved using a Tosoh TSK gel super SW3000 in combination with an Agilent HPLC 1200. The samples were eluted in a 160 mM PBS/200 mM arginine buffer at pH 6.8 at a flowrate of 0.2 ml/min.

The charge variants of 076D-M007-H04-CDRL3-N110D were determined using a Capillary Isoelectric focusing (cIEF). In this method the samples of 076D-M007-H04-CDRL3-N110D were separated in an electrical field (SCIEX PA800 Enhanced, Beckman Coulter) due to their charge while the variants were detected using a UV-vis method. The focusing step of the charge variants was achieved with holding the samples for 15 minutes at 25 kV under normal polarity. The chemical mobilization was conducted holding the samples at 30 kV for 30 minutes. After this procedure the data collection was stopped.

The biochemical test for 076D-M007-H04-CDRL3-N110D was reported as the binding capacity using an Enzyme-Linked Immunosorbant Assay (ELISA). The binding capacity was then compared to a reference standard containing 20 mM L-histidine/50 mM L-arginine hydrochloride/50 mM glycine buffer, 5% trehalose dihydrate and 0.1% polysorbate 80 at pH 5 at <−60° C. The absorption values of reference standard and test samples were compared.

The particle formation was monitored using light obscuration (HIAC, Beckman Coulter) covering a range from 2 μm to 100 μm of particle size. The experiments were conducted with the automated test procedure as a triplicate test for pooled samples from 10 individual vials. For each measurement 5 ml of samples were used.

The determination of turbidity of the solutions was carried out with the aid of a turbidity measurement using the turbidimeter 2100NIS (HachLange, Dusseldorf). 3 ml of 076D-M007-H04-CDRL3-N110D were measured and compared to an optical reference standard in accordance with Ph.Eur. (RSI-IV).

TABLE 15
Stability data of lyophilized 076D-M007-H04-
CDRL3-N110D at 2-8° C.
Test results after reconstitution with sterile water

| Time | | Particle count | Particle count |
|---|---|---|---|
| points | | 10 μm-100 μm | 25 μm-100 μm |
| Months | pH | Particles/ml | Particles/ml |
| 0 | 5.0 | 25 | 1 |
| 3 | 4.9 | 25 | 1 |
| 6 | 4.9 | 29 | 8 |
| 9 | 4.9 | — | — |
| 12 | 5.0 | 21 | 2 |
| 18 | 5.0 | — | — |
| 24 | 5.0 | 53 | 6 |

Table 15 shows the results of the stability study of the lyophilized high-concentration composition 32 of 076D-M007-H04-CDRL3-N110D at 2-8° C. Over a time period of 24 months no significant changes in stability parameters as pH or particulate matter could be observed.

TABLE 163
Stability data of lyophilized 076D-M007-H04-
CDRL3-N110D at 2-8° C.
Further test results after reconstitution with sterile water

| | | | | | Protein |
|---|---|---|---|---|---|
| Time | SEC | | | cIEF | concen- |
| points | HMW | Monomer | Elisa | Main | tration |
| Months | % | % | % | Peak % | mg/ml |
| 0 | 1.2 | 98 | 119 | 74 | 144 |
| 3 | 1.4 | 97 | 128 | 73 | 152 |
| 6 | 1.3 | 98 | 114 | 73 | 151 |
| 9 | 1.5 | 98 | 105 | 72 | 153 |
| 12 | 1.7 | 97 | 91 | 72 | 152 |
| 18 | 1.6 | 97 | 119 | 73 | 150 |
| 24 | 1.7 | 97 | 96 | 69 | 154 |

Table 16 shows further results of the stability study of the lyophilized high-concentration composition 32 of 076D-M007-H04-CDRL3-N110D. Over a time period of 24 months no significant changes in stability parameters as monomeric content, binding capacity, charge variants or protein concentration could be observed.

TABLE 17
Stability data of lyophilized 076D-M007-H04-CDRL3-
N110D at 25° C.
Test results after reconstitution with sterile water.

| Time | | Particle count | Particle count |
|---|---|---|---|
| points | | 10 μm-100 μm | 25 μm-100 μm |
| Months | pH | Particles/ml | Particles/ml |
| 0 | 5.0 | 25 | 1 |
| 3 | 4.9 | 27 | 1 |
| 6 | 4.9 | 42 | 6 |
| 9 | 4.9 | — | — |
| 12 | 4.9 | 16 | 1 |

Table 17 shows the results of the stability study of the lyophilized high-concentration composition 32 of 076D-M007-H04-CDRL3-N110D at 25° C. Over a time period of 12 months no significant changes in stability parameters as pH or particulate matter could be observed.

TABLE 18
Stability data of lyophilized 076D-M007-H04-CDRL3-
N110D at 25° C.
Further test results after reconstitution with sterile water

| | | | | cIEF | Protein |
|---|---|---|---|---|---|
| Time | SEC | | | Main | concen- |
| points | HMW | Monomer | Elisa | Peak | tration |
| Months | % | % | % | % | mg/ml |
| 0 | 1.2 | 98 | 119 | 74 | 144 |
| 3 | 2.6 | 96 | 112 | 71 | 147 |
| 6 | 3.3 | 96 | 101 | 70 | 151 |
| 9 | 4.0 | 95 | 105 | 69 | 154 |
| 12 | 4.7 | 94 | 94 | 68 | 151 |

Table 18 shows further results of the stability study of the lyophilized high-concentration composition 32 of 076D-M007-H04-CDRL3-N110D. Compared to the stability data at 2-8° C. a decrease of the monomeric content from 98 to 94% as well as a shift from 74% to 68% in charge variants (cIEF) could be observed. However the stability parameters as protein concentration and binding capacity showed no significant change in this time period.

Overall composition 32 in lyophilized state was confirmed to be stable upon storage at 2-8° C. for at least 12 months.

### Example 9: Long Term Stability of Liquid High-Concentration Formulation

This example describes the long term stability of liquid high-concentration formulation of 076D-M007-H04-CDRL3-N110D in composition 32 at two different antibody concentrations [150 mg/ml (32) and 100 mg/ml (34)] in comparison to a composition comprising phosphate as buffer (34) instead of amino acids.

076D-M007-H04-CDRL3-N110D was formulated at approximately 150 mg/ml in the following composition:
(32) 20 mM L-Histidine, 50 mM L-Arginine hydrochloride, 50 mM Glycine, 5% trehalose dihydrate, 0.01% Polysorbate 80, pH 5.0
   Or in phosphate-buffer:
(33) 50 mM Phosphate, 5% trehalose dihydrate, 0.1% Polysorbate 80, pH 5.0
   Additionally, 076D-M007-H04-CDRL3-N110D was tested at an antibody concentration of approximately 100 mg/ml in the same buffer system as composition 32:
(34) 20 mM L-Histidine, 50 mM L-Arginine hydrochloride, 50 mM Glycine, 5% trehalose dihydrate, 0.01% polysorbate 80, pH 5.0

The liquid compositions were stored for a time period of 6 month at 2 to 8° C. At specified time points (2, 4 and 6 months) samples were analysed as described above.

Additionally the samples were analysed regarding their IgG purity under reduced conditions (heavy and light chains) using a capillary electrophoresis method (CGE, red.-SCIEX PA 800 Enhanced, Beckman Coulter) to compare the fragmentation of the different embodiments.

As shown in Table 19 all parameters as binding capacity, monomeric content as well as turbidity were stable for a 6 month time period at 2-8° C. in compositions 32 and 34.

TABLE 19
Stability data of liquid 076D-M007-H04-CDRL3-N110D
at 2-8° C. for 6 months

| | | | | Protein | |
|---|---|---|---|---|---|
| Time | Compo- | | | concen- | |
| points | sition | | Turbidity | tration | ELISA |
| Months | No. | pH | NTU | mg/ml | % |
| 0 | 32 | 5.02 | 6.79 | 142.30 | 107 |
| | 33 | 5.31 | 18.70 | 150.42 | 99 |
| | 34 | 4.99 | 7.65 | 102.85 | 96 |
| 2 | 32 | 5.01 | 6.83 | 147.20 | 71 |
| | 33 | 5.46 | 25.70 | 146.15 | 94 |
| | 34 | 4.97 | 8.02 | 102.08 | 105 |
| 4 | 32 | 5.02 | 6.13 | 148.36 | 93 |
| | 33 | — | — | — | 91 |
| | 34 | 4.98 | 7.71 | 101.30 | 88 |
| 6 | 32 | 5.08 | 6.57 | 156.05 | 124 |
| | 33 | 5.61 | 28.20 | 149.38 | 94 |
| | 34 | 5.06 | 8.41 | 104.78 | 98 |

Table 19 shows the results of the stability study of the liquid high-concentration formulations 32-34 of 076D-M007-H04-CDRL3-N110D. The compositions comprising the histidine/glycine/arginine, composition 32 (150 mg/ml 076D-M007-H04-CDRL3-N110D) as well as composition 34 (100 mg/ml 076D-M007-H04-CDRL3-N110D) were stable in terms of pH, turbidity, protein concentration as well as binding capacity.

However, 076D-M007-H04-CDRL3-N110D was not stable at the same storage conditions in phosphate buffer (composition 33). The phosphate buffer containing composition 33 was not able to stabilize the pH of the solution and the turbidity value increased almost to reference standard IV (30 NTU).

TABLE 20
Stability data of liquid 076D-M007-H04-CDRL3-N110D at 2-8° C.
for 6 months

| | | Particle | Particle | | | | |
|---|---|---|---|---|---|---|---|
| | | count | count | cIEF | CGE | | |
| Time | Compo- | 10 μm- | 25 μm- | Main | reduced | SEC | |
| point | sition | 100 μm | 100 μm | Peak | Sum of | Monomer | HMW |
| Months | No. | Particles/ml | | % | H + L % | % | % |
| 0 | 32 | 600 | 222 | 68.66 | 98.60 | 96.05 | 2.92 |
| | 33 | 823 | 190 | 69.26 | 98.65 | 95.25 | 3.73 |
| | 34 | 450 | 68 | 67.26 | 98.35 | 96.12 | 2.84 |
| 2 | 32 | 977 | 160 | 69.54 | — | 96.88 | 2.98 |
| | 33 | 126 | 7 | 69.05 | — | 96.07 | 3.77 |
| | 34 | 257 | 52 | 69.42 | — | 97.01 | 2.85 |
| 4 | 32 | 117 | 71 | 68.67 | 98.18 | 96.01 | 3.03 |
| | 33 | — | — | — | — | — | — |
| | 34 | 135 | 14 | 68.78 | 98.03 | 96.06 | 2.95 |
| 6 | 32 | 18 | 2 | 68.85 | 98.33 | 95.98 | 2.98 |
| | 33 | 2050 | 68 | 53.65 | 96.72 | 92.03 | 5.49 |
| | 34 | 68 | 9 | 68.67 | 98.01 | 96.07 | 2.88 |

Table 20 shows further results of the orienting stability study of the liquid high-concentration formulations 32-34 of 076D-M007-H04-CDRL3-N110D. Composition 32 (150 mg/ml) as well as composition 34 (100 mg/ml) were stable in terms of particulate matter, formation of charge variants (cIEF), fragmentation under reduced conditions (CGE) as well as monomeric content (SEC).

However, 076D-M007-H04-CDRL3-N110D was not stable at the same storage conditions in phosphate buffer (composition 34). The particle formation increased disproportionately compared to compositions comprising the histidine-glycine-arginine buffers. The results of the isoelectric focusing (cIEF) showed that after 6 months the charge variants of 076D-M007-H04-CDRL3-N110D increased in phosphate buffer compared to the histidine/glycine/arginine buffers compositions. Additionally the reduced fragments that were analysed using CGE showed a decrease of the sum of heavy and light chains indicating a fragmentation of the antibody in the phosphate buffer.

The analysis of the long term stability data of the liquid formulation led to the conclusion that composition 32, comprising the histidine-glycine-arginine buffer system according to the invention surprisingly stabilizes high-concentration formulations of 076D-M007-H04-CDRL3-N110D in liquid state for at least 6 months. Lyophilization of the formulation according to the invention is possible but not required as the high-concentration formulation anti-FXIa antibodies according to this invention is stable as liquid formulation over a long period.

If lyophilisation is desired, it should preferably be conducted by the spray-freeze-drying method described herein which provides for a significantly shorter reconstitution time as compared to lyophilizates obtained by conventional freeze-drying or obtained by the process disclosed in WO 2006/008006 A1.

Table 21 and 22 shows further results of the stability study of the liquid high concentration composition 32 of 076D-M007-H04-CDRL3-N110D. Over a time period of 9 months no significant changes in stability parameters as monomeric content, binding capacity, charge variants or protein concentration could be observed.

TABLE 21
Stability data of liquid 076D-M007-H04-CDRL3-N110D at
2-8° C. for 9 months

| | | | | Protein | |
|---|---|---|---|---|---|
| Time | Compo- | | | concen- | |
| points | sition | | Turbidity | tration | ELISA |
| Months | No. | pH | NTU | mg/mL | % |
| 1 | 32 | 5.00 | RS III | 154.60 | 93.96 |
| 3 | 32 | 5.01 | RS III | 153.68 | 91.70 |
| 6 | 32 | 5.06 | RS III | 152.38 | 100.57 |
| 9 | 32 | 4.98 | — | 153.85 | — |

TABLE 22
Stability data of liquid 076D-M007-H04-CDRL3-N110D at 2-8° C.
for 9 months

| | | Particle | Particle | | | | |
|---|---|---|---|---|---|---|---|
| | | count | count | cIEF | CGE | | |
| Time | Compo- | 10 μm- | 25 μm- | Main | reduced | SEC | |
| points | sition | 100 μm | 100 μm | Peak | Sum of | Monomer | HMW |
| Months | No. | Particles/ml | | % | H + L % | % | % |
| 1 | 32 | — | — | 71.17 | 98.85 | 97.80 | 1.11 |
| 3 | 32 | — | — | 75.33 | 98.67 | 97.43 | 1.36 |
| 6 | 32 | 23 | 2 | 71.32 | 97.78 | 97.38 | 1.28 |
| 9 | 32 | — | — | 74.11 | 98.28 | 97.43 | 1.40 |

### Example 10: Long Term Stability of Frozen Bulk of High-Concentration Formulation

This example describes the long term stability of liquid high-concentration formulation of 076D-M007-H04-CDRL3-N110D in the liquid composition (32) over a time period of 18 months at <−60° C.

The liquid composition was stored for a time period of 12 months at <−60° C. At specified time points (1, 2, 3, 6, 9, 12 and 18 months) samples were analysed as described above.

As shown in Table 23 all relevant parameters as pH, charge variants, monomeric content, high molecular content, activity and protein concentration were stable for a 18 months' time period at <−60° C. in composition 32. Overall composition 32 in frozen state was confirmed to be stable upon storage at <−60° C. for at least 18 months.

TABLE 23
Stability data of liquid 076D-M007-H04-CDRL3-N110D
at < −60° C. for 18 months

| Time | | cIEF | SEC | | | Protein |
|---|---|---|---|---|---|---|
| points | | Main | Monomer | HMW | ELISA | Concentration |
| Months | pH | Peak % | % | % | % | mg/ml |
| 0 | 4.9 | 70 | 96 | 2.5 | 101 | 158 |
| 1 | 5.0 | 69 | 96 | 2.7 | 84 | 153 |
| 2 | 5.0 | 69 | 96 | 2.7 | 93 | 154 |
| 3 | 5.0 | 68 | 96 | 2.7 | 104 | 150 |
| 6 | 4.9 | 69 | 97 | 2.7 | 94 | 160 |
| 9 | 5.0 | 69 | 97 | 2.8 | 68 | 158 |
| 12 | 4.9 | 68 | 96 | 2.8 | 100 | 144 |
| 18 | 4.9 | 70 | 96 | 2.6 | 102 | 160 |

1. A stable liquid pharmaceutical formulation comprising anti-FXIa antibody 076D-M007-H04-CDRL3-N110D at a concentration of about 100 mg/ml or more, 10-20 mM histidine, 25-75 mM glycine and 50-75 mM arginine, wherein the formulation has a pH of 4.7-5.3.

2. The liquid pharmaceutical formulation according to claim 1 comprising further ingredients selected from the group consisting of preservatives, carriers, surfactants and stabilizers.

3. The liquid pharmaceutical formulation according to claim 1, wherein the histidine concentration is 20 mM.

4. The liquid pharmaceutical formulation according to claim 1, wherein the glycine concentration is 50 mM.

5. The liquid pharmaceutical formulation according to claim 1, wherein the arginine concentration is 50 mM.

6. The liquid pharmaceutical formulation according to claim 1 comprising 1-10% (w/v) of a stabilizer.

7. The liquid pharmaceutical formulation according to claim 1 comprising 3-7% (w/v) trehalose dihydrate.

8. The liquid pharmaceutical formulation according to claim 1 comprising surfactants at a concentration of 0.005% to 0.2% (w/v).

9. The liquid pharmaceutical formulation according to claim 1, wherein the surfactant is selected from the group consisting of polysorbate 80, polysorbate 20 and poloxamer 188.

10. A stable liquid pharmaceutical formulation comprising anti-FXIa antibody 076D-M007-H04-CDRL3-N110D at a concentration of about 100 mg/ml or more, 20 mM histidine, 50 mM glycine and 50 mM arginine, 5% (w/v) trehalose dihydrate and 0.1% (w/v) polysorbate 80, wherein the formulation has a pH of 5.

11. A stable liquid pharmaceutical formulation comprising anti-FXIa antibody 076D-M007-H04-CDRL3-N110D at a concentration of about 100 mg/ml or more, 20 mM histidine, 50 mM glycine and 50 mM arginine, 5% (w/v) trehalose dihydrate and 0.05% (w/v) polysorbate 20 or poloxamer 188, wherein the formulation has a pH of 5.

12. A stable liquid pharmaceutical formulation comprising anti-FXIa antibody 076D-M007-H04-CDRL3-N110D at a concentration of about 150 mg/ml or more, 20 mM histidine, 50 mM glycine and 50 mM arginine, 5% (w/v) trehalose dihydrate and 0.1% (w/v) polysorbate 80, wherein the formulation has a pH of 5.

13. A stable liquid pharmaceutical formulation comprising anti-FXIa antibody 076D-M007-H04-CDRL3-N110D at a concentration of about 150 mg/ml or more, 20 mM histidine, 50 mM glycine and 50 mM arginine, 5% (w/v) trehalose dihydrate and 0.05% (w/v) polysorbate 20 or poloxamer 188, wherein the formulation has a pH of 5.

14. A Lyophilizate obtainable by freeze-drying a liquid pharmaceutical formulation according to claim 1.

15. The Lyophilizate according to claim 12 obtained by a method comprising the steps of:
a) freezing droplets of a solution comprising an anti-FXIa antibody to form pellets;
b) freeze-drying the pellets;
characterized in that
in step a) the droplets are formed by means of droplet formation of the solution comprising an anti-FXIa antibody into a cooling tower (100) which has a temperature-controllable inner wall surface (110) and an interior temperature below the freezing temperature of the solution
and that
in step b) the pellets are freeze-dried in a rotating receptacle (210) which is housed inside a vacuum chamber (200).

16. A Dosage form comprising a liquid pharmaceutical formulation or a lyophilizate according to claim 1.

17. The Dosage form according to claim 14, wherein the dosage form is a syringe, a vial, a pen device, or an autoinjector.

18. A method of treatment or prophylaxis of thrombotic or thromboembolic disorders comprising administering the liquid pharmaceutical formulation of claim 1 to a subject in need thereof.


SEQUENCE LISTING (2 sequences)

SEQ ID NO: 1 - protein, 449 aa, Artificial Sequence
  heavy chain anti-FXIa abtibody 076D-M007-H04-CDRL3-N110D
EVQLLESGGGLVQPGGSLRLSCAASGFTFSQYGMDWVRQAPGKGLEWVSGIGPSGGSTVYADSVKGRFTISRDNSKNTLYLQMNSLRAEDTAVYYCTRGGPYYYYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 2 - protein, 214 aa, Artificial Sequence
  light chain anti-FXIa antibody 076D-M007-H04-CDRL3-N110D
DIQMTQSPSSLSASVGDRVTITCQASQDISNYLNWYQQKPGKAPKLLIYDASNLETGVPSRFSGSGSGTDFTFTISSLQPEDIATYYCQQADSFPVTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC